(19) United States
(12) Patent Application Publication
(10) Pub. No.: US 2024/0066028 A1
(43) Pub. Date: Feb. 29, 2024

(54) COMPOSITIONS FOR IMPROVED DELIVERY OF CGRP INHIBITORS

(71) Applicant: Pfizer Ireland Pharmaceuticals, Ringaskiddy (IE)

(72) Inventors: Charles M. CONWAY, Cheshire, CT (US); Gene M. DUBOWCHIK, Killingworth, CT (US); Rajesh KUMAR, Skillman, NJ (US)

(21) Appl. No.: 18/255,949

(22) PCT Filed: Dec. 16, 2021
(86) PCT No.: PCT/US21/63648

§ 371 Date: Jun. 5, 2023

Related U.S. Application Data
(60) Provisional application No. 63/126,550, filed on Dec. 17, 2020.

(51) Int. Cl. A61K 31/496; A61K 9/00; A61K 9/48; A61K 9/20

(52) U.S. Cl. CPC A61K 31/496; A61K 9/006; A61K 9/4858; A61K 9/2013

(57) ABSTRACT

Provided is a pharmaceutical formulation in the form of a softgel dosage form including a calcitonin gene-related peptide (CGRP) inhibitor, a lipophilic phase, and at least one lipophilic surfactant. Also provided is a method for increasing bioavailability of a calcitonin gene-related peptide (CGRP) inhibitor in a subject, including orally administering the pharmaceutical formulation to increase the bioavailability of the CGRP inhibitor in the subject.

### CROSS-REFERENCE TO RELATED APPLICATIONS

This application claims priority to U.S. Provisional Application No. 63/126,550 filed Dec. 17, 2020, and all the benefits accruing therefrom under 35 U.S.C. § 119, the contents of which is incorporated herein in its entirety by reference.

### FIELD OF THE INVENTION

The present invention relates to non-irritating, non-toxic compositions providing enhanced bioavailability of an active therapeutic ingredient. Specifically, the present invention relates to compositions containing carbohydrate surfactants for delivery of calcitonin gene-related peptide (CGRP) inhibitors to a subject and methods of their use.

### BACKGROUND OF THE INVENTION

CGRP inhibitors are often combined with various surfactants in pharmaceutical compositions. However, some compositions do not provide optimal delivery of the CGRP inhibitor due to its low oral bioavailability. In addition, some surfactants may be irritating to mucosal membranes. An ideal bioavailability enhancing surfactant will be non-toxic and non-irritable to the skin or mucosal surfaces, and enhance the passage or absorption of the CGRP inhibitor through membrane barriers without damaging the structural integrity and biological function of the membrane and increase bioavailability of the active therapeutic ingredient.

Several approaches to producing rapidly disintegrating or so-called “fast-dispersing” dosage forms have been described. Upon disintegration in the oral cavity, the drug substance is swallowed resulting in pre-gastric absorption and ultimately gastric absorption. Previously described fast-disperse dosage forms provide for the dosage form to disintegrate or dissolve when placed in the mouth in order to promote pre-gastric or gastric absorption of the active ingredient. However, there remains a need in new fast dispersing dosage forms that provide improved characteristics, such as speeding the onset of drug action and reducing the first-pass effect drug metabolism.

### SUMMARY OF THE INVENTION

The present invention is directed to pharmaceutical compositions and methods for increasing bioavailability of a calcitonin gene-related peptide (CGRP) inhibitors.

An embodiment provides a pharmaceutical composition including a CGRP inhibitor and an absorption increasing amount of a carbohydrate surfactant.

Another embodiment provides a pharmaceutical composition including a CGRP inhibitor and an absorption increasing amount of a carbohydrate surfactant, wherein the pharmaceutical composition is in a form of an oral solid molded fast-dispersing dosage form.

Another embodiment provides a pharmaceutical composition comprising a pharmaceutically acceptable carrier and a therapeutically effective amount of zavegepant, a solvate thereof, or a pharmaceutically acceptable salt thereof, wherein the pharmaceutical composition is in a form of an oral solid molded fast-dispersing dosage form.

Another embodiment provides a method for increasing bioavailability of a CGRP inhibitor in a subject, including orally administering any of the above pharmaceutical compositions.

Another embodiment provides a method for treating migraine in a subject in need thereof, including orally administering to the subject any of the above pharmaceutical compositions.

Another embodiment provides a method for providing rapid onset of migraine pain relief in a subject in need thereof, including orally administering to the subject any of the above pharmaceutical compositions.

Another embodiment provides a method for providing a reduced incidence of migraine pain recurrence in a subject in need thereof, including orally administering to the subject any of the above pharmaceutical compositions.

Another embodiment provides a method for treatment or prevention of a condition associated with aberrant levels of CGRP in a subject in need thereof, wherein the method comprises administering to the subject any of the above pharmaceutical compositions.

Another embodiment provides a method for treatment or prevention of a condition associated with aberrant levels of CGRP in a subject in need thereof, wherein the method comprises administering to the subject a pharmaceutical formulation comprising: a synthetic or natural poorly permeable calcitonin gene-related peptide (CGRP) inhibitor or salt or solvate thereof in an amount 0.01-20 weight % of the total weight of the formulation; a lipophilic phase comprising triglycerides of fatty acids in an amount of 50-80 weight % of the total weight of the formulation; and at least one lipophilic surfactant comprising partial esters of polyol and fatty acids in an amount of 10-50 weight % of the total weight of the formulation.

Another embodiment provides a method for treatment or prevention of a condition associated with aberrant levels of CGRP in a subject in need thereof, wherein the method comprises administering to the subject a dosage form comprising: a pharmaceutical formulation comprising: a synthetic or natural poorly permeable CGRP inhibitor or salt or solvate thereof in an amount 0.01-20 weight % of the total weight of the formulation; a lipophilic phase comprising triglycerides of fatty acids in an amount of 50-80 weight % of the total weight of the formulation; and at least one lipophilic surfactant comprising partial esters of polyol and fatty acids in an amount of 10-50 weight % of the total weight of the formulation, wherein the delayed release dosage form is a coated dosage form whose release is pH dependent.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph of plasma concentration (nanograms per milliliter, ng/mL) versus time (hours, hr) showing average BHV-3500 concentration in dog plasma (ng/mL) after a single 50 milligram (mg) sublingual tablet administration;

FIGS. 2 and 3 are graphs of plasma concentration nanograms per milliliter, ng/mL) versus nominal time (hours, hr) showing profiles of the BHV-3500 QD SoftGel 50 mg PK study; and

FIGS. 4 and 5 are graphs of plasma concentration nanograms per milliliter, ng/mL) versus nominal time (day) showing profiles of the BHV-3500 QD SoftGel 50 mg PK food effect study.

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto, but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C1-C10 amine group, a nitro group (—NO₂), a C1-C10 alkyl group, a C3-C10 cycloalkyl group, a C6-C12 aryl group, a Cl-C10 alkoxy group, a C1 to C10 trifluoroalkyl group such as a trifluoromethyl group (—CF₃) and the like, or a cyano group (—CN) instead of at least one hydrogen of a substituting group or compound.

The starting materials useful for making the pharmaceutical compositions of the present invention are readily commercially available or can be prepared by those skilled in the art.

Additional aspects will be set forth in part in the description which follows and, in part, will be apparent from the description.

Embodiment of the present invention are directed to compositions for improved delivery of CGRP inhibitors to the subjects and methods of their use. An embodiment provides a pharmaceutical composition including a calcitonin gene-related peptide (CGRP) receptor antagonist, and an absorption increasing amount of a carbohydrate surfactant.

### CGRP Inhibitors

The composition according to embodiments of the present invention includes a calcitonin gene-related peptide (CGRP) inhibitor. As used herein, the term “CGRP inhibitor” refers to a chemical entity that may be an inhibitor of a CGRP ligand or CGRP receptor. Thus, the term “CGRP inhibitor” encompasses CGRP receptor inhibitors. CGRP (calcitonin gene-related peptide) is a 37 amino acid neuropeptide, which belongs to a family of peptides that includes calcitonin, adrenomedullin, and amylin. Substantial evidence has been collected to show that CGRP is implicated in pathophysiology of migraine. Clinical trials were carried out to prove that CGRP inhibitors are effective in treating migraine. Following clinical trials, several CGRP inhibitors have been approved by regulatory authorities and marketed for treatment of acute migraine and migraine prevention.

The CGRP inhibitor may be a CGRP antibody, a CGRP receptor antibody, an antigen-binding fragment from a CGRP antibody or a CGRP receptor antibody, a CGRP infusion inhibitory protein, a CGRP bio-neutralizing agent, a small molecule CGRP receptor antagonist, a small molecule CGRP inhibitor, or a polypeptide CGRP inhibitor.

The CGRP inhibitor may be a CGRP receptor antagonist. The CGRP receptor antagonists in accordance with the present invention are preferably non-biologic CGRP antagonists. That is, the non-biologic CGRP receptor antagonists of the present invention preferably do not contain antibodies, antibody fragments, or peptides. The CGRP receptor antagonist may be a small molecule receptor antagonist. Preferably, the small molecule CGRP receptor antagonists in accordance with the present invention contain molecules with a mass of less than about 900 Daltons, for example, less than about 800 Daltons, less than about 700 Daltons, less than about 600 Daltons, less than about 500 Daltons, less than about 400 Daltons, or less than about 300 Daltons. Examples of such non-biologic CGRP antagonists include, rimegepant, zavegepant, ubrogepant, atogepant, telcagepant, and olcegepant. In an embodiment, the small molecule CGRP receptor antagonist may be (R)-N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide (zavegepant).

Rimegepant has the chemical formula, C₂₈H₂₈F₂N₆O₃ and the IUPAC name [(5S,6S,9R)-5-amino-6-(2,3-difluorophenyl)-6,7,8,9-tetrahydro-5H-cyclohepta[b]pyridin-9-yl] 4-(2-oxo-3H-imidazo[4,5-b]pyridin-1-yl)piperidine-1-carboxylate. Rimegepant is also known as and referred to herein as BHV-3000.

The structure of rimegepant is:

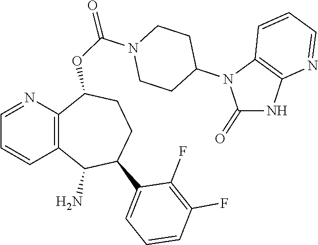 [SMILES: N[C@@H]1c2cccnc2[C@H](OC(=O)N2CCC(n3c(=O)[nH]c4ncccc43)CC2)CC[C@H]1c1cccc(F)c1F]

Rimegepant is described, for example, in WO 2011/046997 published Apr. 21, 2011. In a preferred aspect of the invention, rimegepant is present in the form of a hemisulfate sesquihydrate salt. This preferred salt form is described in WO 2013/130402 published Sep. 6, 2013.

The chemical formula of the salt form is C₂₈H₂₈F₂N₆O₃.0.5 H₂SO₄.1.5 H₂O and the structure is as follows:

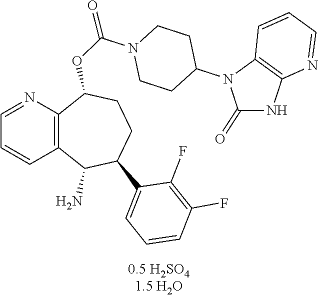 [SMILES: N[C@@H]1c2cccnc2[C@H](OC(=O)N2CCC(n3c(=O)[nH]c4ncccc43)CC2)CC[C@H]1c1cccc(F)c1F]

Another CGRP antagonist is zavegepant (also known as BHV-3500), which is described in WO 2011/123232 published Oct. 6, 2011, and has the following structure:

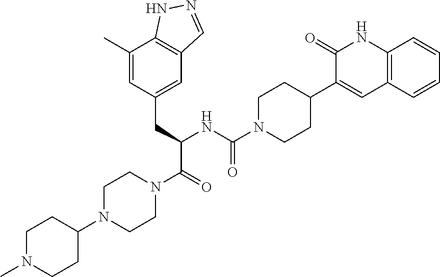 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Another CGRP antagonist is ubrogepant, which has the following structure:

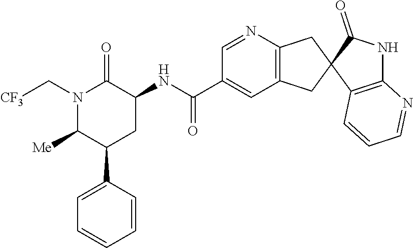 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

Another CGRP antagonist is atogepant, which has the following structure:

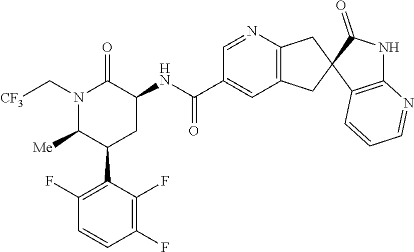 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

Another CGRP antagonist is telcagepant, which has the following structure:

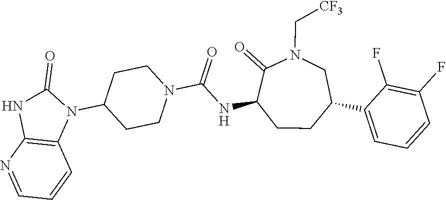 [SMILES: O=C(N[C@@H]1CC[C@@H](c2cccc(F)c2F)CN(CC(F)(F)F)C1=O)N1CCC(n2c(=O)[nH]c3ncccc32)CC1]

Another CGRP antagonist is olcegepant, which has the following structure:

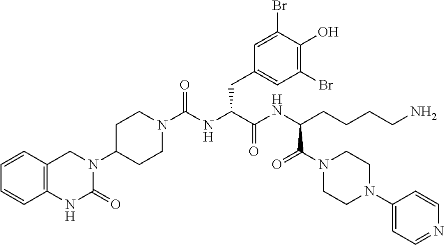 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

The CGRP inhibitor may have low oral bioavailability. The low oral bioavailability may be 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less.

The compositions described herein may include 1-1000 mg of the CGRP inhibitor. For example, the compositions may include about 1, 5, 10, 15, 20, 25, 30, 40, 50, 60, 70, 80, 90, 100, 200, 250, 300, 400, 500, 600, 700, 800, or 900 mg of the CGRP inhibitor. The amount of the CGRP inhibitor may range between any of the above values.

The CGRP inhibitor may be administered at a dose of about 1-1000 mg per day. In another aspect, the CGRP inhibitor is administered at a dose of about 1, 5, 10, 15, 20, 25, 30, 40, 50, 60, 70, 80, 90, 100, 200, 250, 300, 400, 500, 600, 700, 800, or 900 mg per day. The daily dose of the CGRP inhibitor may range between any of the above values. The daily dose of the CGRP inhibitor may range between any of the above values. The composition including a CGRP inhibitor may be administered as a single dose.

The CGRP inhibitor may be administered for at least one week and for as long as needed. For example, the CGRP inhibitor may be administered for one week, two weeks, three weeks, four weeks, five weeks, six weeks, seven weeks, eight weeks, nine weeks, ten weeks, eleven weeks, or twelve weeks.

### Carbohydrate Surfactants

The composition further includes a carbohydrate surfactant in an absorption increasing amount. As used herein, a “carbohydrate” is inclusive of monocarbohydrates, oligocarbohydrates, or polycarbohydrates in straight chain or ring forms, or a combination thereof to form a carbohydrate chain. Oligocarbohydrates are carbohydrates having two or more but less than 100 monohydrate residues. Polycarbohydrates include 100 or more monohydrates residues. The carbohydrate may be chosen, for example, from any currently commercially available monocarbohydrate species or may be synthesized. Some examples of the many possible carbohydrates to use include glucose, maltose, maltotriose, maltotetraose, sucrose, and trehalose. Preferable carbohydrates include maltose, sucrose, and glucose.

In an embodiment, the carbohydrate surfactant may be an alkyl glycoside. As used herein, the term “alkyl glycoside” refers to any carbohydrate joined by a linkage to any hydrophobic alkyl, as is known in the art. The alkyl glycoside is preferably non-toxic and non-ionic, and increases the absorption of a CGRP inhibitor when it is administered with the compound via the oral, ocular, intranasal, nasolacrimal, nose-to-brain, inhalation or pulmonary, oral cavity (sublingual or Buccal cell), or cerebrospinal fluid (CSF) delivery route. Suitable compounds can be determined using the methods set forth herein.

Alkyl glycosides disclosed herein may be synthesized by known procedures, i.e., chemically, as described, for example, in Rosevear et al., Biochemistry 19: 4108-4115 (1980) or Koeltzow and Urfer, J. Am. Oil Chem. Soc., 61: 1651-1655 (1984), U.S. Pat. No. 3,219,656 and 3,839,318 or enzymatically, as described, for example, in Li et al., J. Biol. Chem., 266: 10723-10726 (1991) or Gopalan et al., J. Biol. Chem. 267: 9629-9638 (1992).

Alkyl glycosides of the present invention may include, but are not limited to: alkyl glycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside (synthesized according to Koeltzow and Urfer; Anatrace Inc., Maumee, Ohio; Calbiochem, San Diego, Calif.; Fluka Chemie, Switzerland); alkyl thiomaltosides, such as heptyl, octyl, dodecyl-, tridecyl-, and tetradecyl-β-D-thiomaltoside (synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144: 7-11 (1980)); alkyl thioglucosides, such as heptyl- or octyl 1-thio α- or βP-D-glucopyranoside (Anatrace, Inc., Maumee, Ohio; see Saito, S, and Tsuchiya, T. Chem. Pharm. Bull. 33: 503-508 (1985)); alkyl thiosucroses (synthesized according to, for example, Binder, T. P. and Robyt, J. F., Carbohydr. Res. 140: 9-20 (1985)); alkyl maltotriosides (synthesized according to Koeltzow and Urfer); long chain aliphatic carbonic acid amides of sucrose β-amino-alkyl ethers; (synthesized according to Austrian Patent 382,381 (1987); Chem. Abstr., 108: 114719 (1988) and Gruber and Greber pp. 95-116); derivatives of palatinose and isomaltamine linked by amide linkage to an alkyl chain (synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (synthesized according to Kunz); long chain aliphatic carbonic acid ureides of sucrose β-amino-alkyl ethers (synthesized according to Gruber and Greber, pp. 95-116); and long chain aliphatic carbonic acid amides of sucrose β-amino-alkyl ethers (synthesized according to Austrian Patent 382,381 (1987), Chem. Abstr., 108: 114719 (1988) and Gruber and Greber, pp. 95-116).

In another embodiment, the carbohydrate surfactant may be a carbohydrate ester. As used herein, the term “carbohydrate ester” refers to a carbohydrate ester of any fatty acids. Carbohydrate esters may take many forms because of several hydroxyl groups in the carbohydrate are available for reaction and the many fatty acid groups, from acetate on up to larger, more bulky fatty acids that may be reacted with the carbohydrate. This flexibility means that many products and functionalities may be tailored, based on the fatty acid moiety used. Carbohydrate esters have food and non-food uses, especially as surfactants and emulsifiers, with growing applications in pharmaceuticals, cosmetics, detergents and food additives. They are biodegradable, non-toxic, and mild to the skin. In an embodiment, the carbohydrate ester may be a sucrose ester.

The carbohydrate surfactants disclosed herein may have a hydrophobic alkyl group linked to a hydrophilic carbohydrate. The linkage between the hydrophobic alkyl group and the hydrophilic carbohydrate may include, among other possibilities, a glycosidic, thioglycosidic (Horton), amide (Carbohydrates as Organic Raw Materials, F. W. Lichtenthaler ed., VCH Publishers, New York, 1991), ureide (Austrian Patent 386,414 (1988); Chem. Abstr. 110: 137536p (1989); see Gruber, H. and Greber, G., “Reactive Sucrose Derivatives” in Carbohydrates as Organic Raw Materials, pp. 95-116) or ester linkage (Sugar Esters: Preparation and Application, J. C. Colbert ed., (Noyes Data Corp., New Jersey), (1974)). Further, preferred glycosides may include maltose, sucrose, and glucose linked by glycosidic linkage to an alkyl chain of about 9-16 carbon atoms, e.g., nonyl-, decyl-, dodecyl- and tetradecyl sucroside, glucoside, and maltoside, but are not limited thereto. These compositions are amphipathic and non-toxic, because they degrade to an alcohol and an oligocarbohydrate.

In another embodiment, carbohydrate surfactants may include an alkyl glycoside and/or a carbohydrate ester having characteristic hydrophile-lipophile balance (HLB) numbers, which may be calculated or determined empirically (Schick, M. J. Nonionic Surfactants, p. 607 (New York: Marcel Dekker, Inc. (1967)). The HLB number is a direct reflection of the hydrophilic character of the surfactant, i.e., the larger the HLB number, the more hydrophilic the compound. HLB numbers may be calculated by the formula: (20 times MW hydrophilic component)/(MW hydrophobic component+MW hydrophilic component), where MW=molecular weight (Rosen, M. J., Surfactants and Interfacial Phenomena, pp. 242-245, John Wiley, New York (1978)). The HLB number is a direct expression of the hydrophilic character of the surfactant, i.e., the larger the HLB number, the more hydrophilic the compound. The carbohydrate surfactant may have an HLB number of from about 10 to 20, for example, from about 11 to 15.

As described above, the hydrophobic alkyl may be chosen of any desired size, depending on the hydrophobicity desired and the hydrophilicity of the carbohydrate moiety. In an embodiment, a range of alkyl chains may be from about 9 to about 24 carbon atoms, for example, from about 9 to about 16 or about 14 carbon atoms. Some glycosides may include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, but are not limited thereto. These compositions are non-toxic, since they are degraded to an alcohol and an oligocarbohydrate, and amphipathic.

The above examples are illustrative of the types of glycosides to be used in the invention claimed herein, but the list is not exhaustive. Derivatives of the above compounds, which fit the criteria of the claims, should also be considered when choosing a glycoside. All of the compounds may be screened for efficacy following the methods taught herein and in the examples.

### Formulations and Methods of Administration

The compositions, according to embodiments of the present invention, may be administered as a tablet, a capsule, a suppository, a drop, a spray, or an aerosol. The compositions may be administered as oral fast-disintegrating tablets. The compositions may also be administered in a sustained release or delayed burst format. The spray and the aerosol administration may be achieved through use of an appropriate dispenser. The sustained release format may be an ocular insert, erodible microparticulates, swelling mucoadhesive particulates, pH sensitive microparticulates, nanoparticles/latex systems, ion-exchange resins and other polymeric gels and implants (Ocusert, Alza Corp., California; Joshi, A., S. Ping and K. J. Himmelstein, Patent Application WO 91/19481). These systems maintain prolonged drug contact with the absorptive surface preventing washout and non-productive drug loss. The prolonged drug contact is non-toxic to the skin and mucosal surfaces.

The compositions disclosed herein are stable. For example, Baudys et al. in U.S. Pat. No. 5,726,154 show that calcitonin in an aqueous liquid composition including SDS (sodium dodecyl sulfate, a surfactant) and an organic acid is stable for at least 6 months. Similarly, the surfactant compositions of the present invention have improved stabilizing characteristics when admixed with a CGRP inhibitor. No organic acid is required in these formulations. For example, the composition may maintain the stability of CGRP inhibitor for about 6 months, or more, when maintained at about 4° C. to 25° C.

The stability of the compositions disclosed herein are, in part, due to their high no observable adverse effect level (NOAEL). The Environmental Protection Agency (EPA) defines the no observable adverse effect level (NOAEL) as the exposure level at which there are no statistically or biologically significant increases in the frequency or severity of adverse effects between the exposed population and its appropriate control. Hence, the term “no observable adverse effect level” (or NOAEL) is the greatest concentration or amount of a substance, found by experiment or observation, which causes no detectable adverse alteration of morphology, functional capacity, growth, development, or life span of the target organism under defined conditions.

The Food and Agriculture Organization (FAO) of the United Nations of the World Health Organization (WHO) has shown that some alkyl glycosides have very high NOAELs, allowing for increased consumption of these alkyl glycosides without any adverse effect. This report can be found on the world wide web at inchem.org/documents/jecfa/jecmono/v10je11.htm. For example, the NOAEL for sucrose dodecanoate, a sucrose ester used in food products, is about 20-30 grams/kilogram/day, e.g., a 70 kilogram person (about 154 pounds) can consume about 1400-2100 grams (or about 3 to 4.6 pounds) of sucrose dodecanoate per day without any observable adverse effect. Typically, an acceptable daily intake for humans is about 1% of the NOAEL, which translates to about 14-21 grams, or 14 million micrograms to 21 million micrograms, per day, indefinitely. Definitions of NOAELs and other related definitions may be found on the world wide web at epa.gov/OCEPAterms. Thus, although some effects may be produced with alkyl glycoside levels anticipated in the present invention, the levels are not considered adverse, or precursors to adverse effects.

Accordingly, a subject treated with the compositions, according to embodiments of the invention, having at least one alkyl glycoside, e.g., tetradecylmaltoside (TDM; or Intravail A), at a concentration of about 0.125% by weight of alkyl glycoside two times per day, or three times per day, or more depending on the treatment regimen consumes about 200 to 300 micrograms per day total of TDM. So, the effective dose of the TDM is at least 100-fold lower than (i.e., 1/1000) of the NOAEL, and falls far below 1% of the NOAEL, which is the acceptable daily intake; or in this case about 1/50,000 of the acceptable daily intake. Stated another way, alkyl glycosides disclosed herein have a high NOAEL, such that the amount or concentration of alkyl glycosides used do not cause an adverse effect and can be safely consumed without any adverse effect.

The compositions, according to embodiments of the invention, are also stable because they are physiologically non-toxic and non-irritants. As used herein, the term “non-toxic” means that the alkyl glycoside molecule has a sufficiently low toxicity to be suitable for human administration and consumption. Preferred alkyl glycosides are non-irritating to the tissues to which they are applied. Any alkyl glycoside used should be of minimal or no toxicity to the cell, such that it does not cause damage to the cell. Yet, toxicity for any given alkyl glycoside may vary with the concentration of alkyl glycoside used. It is also beneficial if the alkyl glycoside chosen is metabolized or eliminated by the body, and if this metabolism or elimination is done in a manner that will not be harmfully toxic. As used herein, the term “non-irritant” means that the agent does not cause inflammation following immediate, prolonged or repeated contact with the skin surface or mucous membranes.

The compositions disclosed herein are typically present in an amount from about 0.01% to 20% by weight based on 100% of the composition weight. For example, the compositions may be present in an amount from about 0.01% to 5% by weight, from about 0.01% to 2% by weight, from about 0.01% to 1%, or from about 0.01% to 0.125% by weight based on 100% of the composition weight. The carbohydrate surfactant may be formulated to be compatible with other components present in the composition. In liquid, or gel, or capsule, or injectable, or spray compositions the carbohydrate surfactant may be formulated such that it promotes, or at least does not degrade, the stability of the CGRP inhibitor. Further, the compositions optimize the concentration by keeping the concentration of absorption enhancer as low as possible, while still maintaining the desired effect.

The compositions disclosed herein, when administered to the subject, yield enhanced mucosal delivery of the CGRP inhibitor with a peak concentration (or Cₘₐₓ) in a tissue, or fluid, or in a blood plasma of the subject that is about 1.15%, 1.25%, 1.50%, 1.75%, 2%, 3%, 4%, 5%, 10%, 15%, 20%, 25%, 30%, 35%, 40%, 45%, or 50% or greater as compared to a Cₘₐₓ of the compound(s) in a tissue (e.g., central nervous system or CNS), or fluid, or blood plasma following intramuscular injection of an equivalent concentration of the compound(s) to the subject.

The measure of how much of the CGRP inhibitor reaches the bloodstream in a set period of time, e.g., 24 hours can also be calculated by plotting drug blood concentration at various times during a 24-hour or longer period and then measuring the area under the curve (AUC) between 0 and 24 hours. Similarly, a measure of drug efficacy may also be determined from a time to maximal concentration (Tₘₐₓ) of the biologically active compound(s) in a tissue (e.g., CNS) or fluid or in the blood plasma of the subject between about 0.1 to 1.0 hours. The therapeutic compositions disclosed herein increase the speed of onset of drug action (i.e., reduce Tₘₐₓ) by a factor of about 1.5-fold or greater, for example, about 1.5 to about 5-fold, about 1.5 to about 4-fold, about 1.5 to about 3-fold, or about 1.5 to about 2-fold.

Also, the therapeutic compositions or formulations, according to embodiments of the invention, can be administered or delivered to a subject in need systemically or locally. Suitable routes may, for example, include oral, ocular, nasal, nose-to-brain, nasolacrimal, inhalation or pulmonary, oral cavity (sublingual or Buccal cell), transmucosal administration, vaginal, rectal, parenteral delivery, including intramuscular, subcutaneous, intravenous, intraperitoneal, or CSF delivery. Moreover, the mode of delivery, e.g., liquid, gel, tablet, spray, etc. will also depend on the method of delivery to the subject.

Additionally, the therapeutic compositions, according to embodiments of the invention, may include a pharmaceutically acceptable carrier. As used herein, the term “pharmaceutically acceptable carrier” refers to an aqueous or non-aqueous agent, for example alcoholic or oleaginous, or a mixture thereof, which may contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well-known in the art and include, for example, aqueous solutions such as water or physiologically buffered saline or other solvents or vehicles such as glycols, glycerol, and oils such as olive oil or injectable organic esters. A pharmaceutically acceptable carrier may contain physiologically acceptable compounds that act, for example, to stabilize or to increase the absorption of the specific inhibitor, for example, carbohydrates, such as glucose, sucrose or dextrans, antioxidants, such as ascorbic acid or glutathione, chelating agents, low molecular weight proteins or other stabilizers or excipients. A pharmaceutically acceptable carrier may also be selected from substances such as distilled water, benzyl alcohol, lactose, starches, talc, magnesium stearate, polyvinylpyrrolidone, alginic acid, colloidal silica, titanium dioxide, and flavoring agents.

Additionally, to decrease susceptibility of alkyl carbohydrates or carbohydrate alkyl esters to hydrolytic cleavage of the CGRP inhibitor, various oxygen atoms within the alkyl carbohydrate or the carbohydrate alkyl ester may be substituted for by sulfur (Defaye, J. and Gelas, J. in Studies in Natural Product Chemistry (Atta-ur-Rahman, ed.) Vol. 8, pp. 315-357, Elsevier, Amsterdam, 1991). For example, the heteroatom of the carbohydrate ring may be either oxygen or sulfur, or the linkage between monocarbohydrate residues in an oligocarbohydrate may be oxygen or sulfur (Horton, D. and Wander, J. D., “Thio Sugars and Derivatives,” The Carbohydrates: Chemistry and Biochemistry, 2d. Ed. Vol. IB, (W. Reyman and D. Horton eds.), pp. 799-842, (Academic Press, New York), (1972)). Oligocarbohydrates may have either α (alpha) or β (beta) anomeric configuration (see Pacsu, E., et al. in Methods in Carbohydrate Chemistry (R. L. Whistler, et al., eds.) Vol. 2, pp. 376-385, Academic Press, New York 1963).

A composition, according to embodiments of the invention, may be prepared in tablet form by mixing a CGRP inhibitor and one alkyl glycoside and/or carbohydrate alkyl ester, and an appropriate pharmaceutical carrier or excipient, for example mannitol, corn starch, polyvinylpyrrolidone or the like, granulating the mixture and finally compressing it in the presence of a pharmaceutical carrier such as corn starch, magnesium stearate or the like. If desired, the formulation thus prepared may include a sugar-coating or enteric coating or covered in such a way that the active principle is released gradually, for example, in the appropriate pH medium.

The term “enteric coating” is a polymer encasing, surrounding, or forming a layer, or membrane around the therapeutic composition or core. Also, the enteric coating may contain a CGRP inhibitor which is compatible or incompatible with the coating. In an example, a tablet composition may include an enteric coating polymer with a compatible CGRP inhibitor which dissolves or releases the inhibitor at higher pH levels (e.g., pH greater than 4.0, greater than 4.5, greater than 5.0 or higher) and not at low pH levels (e.g., pH 4 or less); or the reverse.

In an embodiment, the dependent release form of the invention may be a tablet including:

(a) a core including:
(i) a CGRP inhibitor; and
(ii) a surfactant including at least one alkyl glycoside and/or carbohydrate alkyl ester;

and

(b) at least one membrane coating surrounding the core,

wherein the coating is an impermeable, permeable, semi-permeable, or porous coating, and becomes more permeable or porous upon contacting an aqueous environment of a defined pH.

As used herein, the term “membrane” is synonymous with “coating,” or equivalents thereof. The terms are used to identify a region of a medicament, for example, a tablet, that is impermeable, permeable, semi-permeable or porous to an aqueous solution(s) or bodily fluid(s), and/or to the therapeutic agent(s) or drug(s) encapsulated therein. If the membrane is permeable, semi-permeable or porous to the CGRP inhibitor, the inhibitor may be released through the openings or pores of the membrane in solution or in vivo. The porous membrane may be manufactured mechanically (e.g., drilling microscopic holes or pores in the membrane layer using a laser), or it may be imparted due to the physiochemical properties of the coating polymer(s). Membrane or coating polymers of the invention are well-known in the art, and include cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, and 4,036,228.

Further, the enteric coating described herein may include a plasticizer, and a sufficient amount of sodium hydroxide (NaOH) to effect or adjust the pH of the suspension in solution or in vivo. Examples of plasticizers include triethyl citrate, triacetin, tributyl sebecate, or polyethylene glycol. Other alkalizing agents, including potassium hydroxide, calcium carbonate, sodium carboxymethylcellulose, magnesium oxide, and magnesium hydroxide can also be used to effect or adjust the pH of the suspension in solution or in vivo.

Accordingly, in an embodiment, an enteric coating may be designed to release a certain percentage of a CGRP inhibitor in certain mediums with a certain pH or pH range. For example, the composition, according to embodiment of the invention, may include at least one enteric coating encasing or protecting at least one CGRP inhibitor, which is chemically unstable in an acidic environment (e.g., the stomach). The enteric coating protects the CGRP inhibitor from the acidic environment (e.g., pH<3), while releasing the inhibitor in locations which are less acidic, for example, regions of the small and large intestine where the pH is 3, or 4, or 5, or greater. A medicament of this nature will travel from one region of the gastrointestinal tract to the other, for example, it takes about 2 to about 4 hours for a CGRP inhibitor to move from the stomach to the small intestine (duodenum, jejunum and ileum). During this passage or transit, the pH changes from about 3 (e.g., stomach) to 4, or 5, or to about a pH of 6 or 7 or greater. Thus, the enteric coating allows the core containing the CGRP inhibitor to remain substantially intact, and prevents premature release of the inhibitor or the acid from penetrating and de-stabilizing the CGRP inhibitor.

Examples of suitable enteric polymers include, but are not limited to, cellulose acetate phthalate, hydroxypropylmethylcellulose phthalate, polyvinylacetate phthalate, methacrylic acid copolymer, shellac, cellulose acetate trimellitate, hydroxypropylmethylcellulose acetate succinate, hydroxypropylmethylcellulose phthalate, cellulose acetate phthalate, cellulose acetate succinate, cellulose acetate malate, cellulose benzoate phthalate, cellulose propionate phthalate, methylcellulose phthalate, carboxymethylethylcellulose, ethylhydroxyethylcellulose phthalate, shellac, styrene-acrylic acid copolymer, methyl acrylate-acrylic acid copolymer, methyl acrylate-methacrylic acid copolymer, butyl acrylate-styrene-acrylic acid copolymer, methacrylic acid-methyl methacrylate copolymer, methacrylic acid-ethyl acrylate copolymer, methyl acrylate-methacrylic acid-octyl acrylate copolymer, vinyl acetate-maleic acid anhydride copolymer, styrene-maleic acid anhydride copolymer, styrene-maleic acid monoester copolymer, vinyl methyl ether-maleic acid anhydride copolymer, ethylene-maleic acid anhydride copolymer, vinyl butyl ether-maleic acid anhydride copolymer, acrylonitrile-methyl acrylate-maleic acid anhydride copolymer, butyl acrylate-styrene-maleic acid anhydride copolymer, polyvinyl alcohol phthalate, polyvinyl acetal phthalate, polyvinyl butylate phthalate and polyvinyl acetoacetal phthalate, or combinations thereof. One skilled in the art will appreciate that other hydrophilic, hydrophobic and enteric coating polymers may be readily employed, either alone or in any combination, as all or part of a coating, according to embodiments of the invention.

The therapeutic compositions of the invention in the form of a tablet may have a plurality of coatings, for example, a hydrophilic coating (e.g., hydroxypropylmethylcellulose), and/or a hydrophobic coating (e.g., alkylcelluloses), and/or an enteric coating. For example, the tablet core may be encases by a plurality of the same type of coating, or a plurality of different types of coating selected from a hydrophilic, hydrophobic or enteric coating. Hence, it is anticipated that a tablet may be designed having at least one, but can have more than one layer consisting of the same or different coatings dependent on the target tissue or purpose of the CGRP inhibitor. For example, the tablet core layer may have a first composition enclosed by a first coating layer (e.g., hydrophilic, hydrophobic, or enteric coating), and a second same or different composition or CGRP inhibitor having the same or different dosage may be enclosed in second coating layer, etc. This layering of various coatings provides for a first, second, third, or more gradual or dose dependent release of the same or different CGRP inhibitor containing composition.

In an embodiment, a first dosage of a first composition of the invention is contained in a tablet core and with an enteric-coating, such that the enteric-coating protects and prevents the composition contained therein from breaking down or being released into the stomach. In another example, the first loading dose of the therapeutic composition is included in the first layer and includes from about 10% to about 40% of the total amount of the total composition included in the formulation or tablet. In a second loading dose, another percentage of the total dose of the composition is released. The invention contemplates as many time release doses as desired in a treatment regimen. Thus, in certain aspects, a single coating or plurality of coating layers may be in an amount ranging from about 2% to 6% by weight, for example, about 2% to about 5%, for example, from about 2% to about 3% by weight of the coated unit dosage form.

Accordingly, the formulations according to embodiments of the invention make it possible for contents of a hard capsule or tablet to be selectively released at a desired site the more distal parts of the gastro-intestinal tract (e.g., small and large intestine) by selecting a suitable pH-soluble polymer for a specific region. Mechanical expulsion of the composition preparations may also be achieved by inclusion of a water absorbing polymer that expands upon water absorption within a hard semi-permeable capsule, thus expelling composition through an opening in the hard capsule.

Further, it will be understood by one of ordinary skill in the art, that the specific dose level and frequency of dosage for any particular subject in need of treatment may be varied and will depend upon a variety of factors including the activity of the specific CGRP inhibitor employed, the metabolic stability, and length of action of that compound, the age, body weight, general health, sex, diet, mode and time of administration, rate of excretion, drug combination, the severity of the particular condition, and the host undergoing therapy.

Alkyl glycosides, for example, alkylmaltosides, for example, dodecyhnaltoside (DDM) and tetradecylmaltoside (TDM), may stabilize the CGRP inhibitor in solution and prevent its aggregation. Accordingly, an aspect of the invention is to provide therapeutic compositions having at least one CGRP inhibitor and one carbohydrate surfactant, wherein the surfactant further includes at least one alkyl glycoside and/or carbohydrate alkyl ester formulation, which enhances the bioavailability of the CGRP inhibitor. Determining the bioavailability of drug formulations is described herein. As used herein, “bioavailability” is the rate and extent to which the active substance, or moiety, which reaches the systemic circulation as an intact drug. The bioavailability of any drug will depend on how well it is adsorbed and how much of it escapes removal by the liver.

To determine absolute bioavailability, the tested drug and mode of administration is measured against an intravenous reference dose. The bioavailability of the intravenous dose is 100% by definition. For example, animals or volunteering humans are given an intravenous injections and corresponding oral doses of a drug. Urinary or plasma samples are taken over a period of time and levels of the drug over that period of time are determined.

The areas under the curve (AUC), of the plasma drug concentration versus time curves, are plotted for both the intravenous and the oral doses, and calculation of the bioavailability of both formulations is by simple proportion. For example, if the same intravenous and oral doses are given, and the oral AUC is 50% of the intravenous AUC, the bioavailability of the oral formulation is 50%. Indeed, the bioavailability of any drug is due to many factors including incomplete absorption, first pass clearance or a combination of these (discussed more below). Further, the peak concentration (or Cₘₐₓ) of the plasma drug concentration is also measured to the peak concentration (Cₘₐₓ) of the plasma drug concentration following intramuscular (IM) injection of an equivalent concentration the drug. Moreover, the time to maximal concentration (or Tₘₐₓ) of the plasma drug is about 0.1 to 1.0 hours.

To determine the relative bioavailability of more than one formulation of a drug (e.g., an alkyl glycoside or carbohydrate alkyl ester drug formulation), bioavailability of the formulations are assessed against each other as one or both drugs could be subject to first pass clearance (discussed more below), and thus, undetected. For example, a first oral formulation is assessed against a second oral formulation. The second formulation is used as a reference to assess the bioavailability of the first. This type of study provides a measure of the relative performance of two formulations in getting a drug absorbed.

The alkyl glycosides or carbohydrate, according to embodiments of the present invention, include any compounds now known or later discovered. CGRP inhibitors which are particularly well-suited for admixture with the alkyl glycosides and/or carbohydrate alkyl esters are those that are difficult to administer by other methods, e.g., compounds that are degraded in the gastrointestinal (GI) tract or those that are not absorbed well from the GI tract, or compounds that can be self-administered via the oral, ocular, nasal, nasolacrimal, inhalation, sublingual, or CSF delivery route instead of traditional methods such as injection.

Alternatively, bioavailability of a CGRP inhibitor can be determined by measuring the levels of the drug's first pass clearance by the liver. Alkyl glycosides and/or carbohydrate alkyl ester compositions of the invention administered intranasally or via oral cavity (sublingual or Buccal cell) do not enter the hepatic portal blood system, thereby avoiding first pass clearance by the liver. Avoiding first past clearance of these formulations by the liver is described herein. As used herein, the term, “first pass liver clearance” is the extent to which the drug is removed by the liver during its first passage in the portal blood through the liver to the systemic circulation. This is also called first pass metabolism or first pass extraction.

The two major routes of drug elimination from the body are excretion by the kidneys whereby the drug is unchanged; and elimination by the liver, whereby the drug is metabolized. The balance between these two routes depends on the relative efficiency of the two processes. The present invention describes herein elimination by the liver or liver clearance. First pass liver clearance is described by Birkett et al (1990 and 1991), which is incorporated by reference in its entirety. Birkett et al., Aust Prescr, 13 (1990): 88-9; and Birkett et al., Austra Prescr, 14: 14-16 (1991).

Blood carrying drug from the systemic circulation enter the liver via the portal vein, and the liver in turn extracts a certain percentage or ratio (i.e., 0.5 or 50%) of that drug. The remainder left over (i.e., 0.2 or 20%) re-enters the systemic circulation via the hepatic vein. This rate of clearance of the drug is called the hepatic extraction ratio. It is the fraction of the drug in the blood which is irreversibly removed (or extracted) during the first pass of the blood through the liver. If no drug is extracted, the hepatic extraction ratio is zero. Conversely, if the drug is highly extracted in the first pass through the liver, the hepatic extraction ratio may be as high as 100% or 1.0. In general, clearance of the drug by the liver depends then on the rate of delivery of that drug to the liver (or the hepatic blood flow), and on the efficiency of removal of that drug (or the extraction ratio).

Therefore, the net equation used to determine hepatic clearance is:

(hepatic clearance−blood flow)=(unbound fraction*intrinsic clearance)/blood flow+(unbound fraction*intrinsic clearance) (1)

The “unbound fraction” of drug is dependent on how tightly the drug is bound to proteins and cells in the blood. In general, it is only this unbound (or free) drug which is available for diffusion from the blood into the liver cell. In the absence of hepatic blood flow and protein binding, the “intrinsic clearance” is the ability of the liver to remove (or metabolize) that drug. In biochemical terms, it is a measure of liver enzyme activity for a particular drug substrate. Again, although intrinsic clearance can be high, drugs cannot be cleared more rapidly than that presented to the liver. In simple terms, there are two situations: where liver enzyme activity is very high or very low (i.e., high extraction ratio or low extraction ratio).

When liver enzyme activity is low, the equation simplifies to:

hepatic clearance=unbound fraction*intrinsic clearance (2)

Clearance then is independent of blood flow, but instead depends directly on the degree of protein binding in the blood and the activity of drug metabolizing enzymes towards that drug.

In contrast, when liver enzyme activity is high, the equation is:

hepatic clearance=liver blood flow (3)

In this scenario, because the enzymes are so active the liver removes most of the drug presented to it and the extraction ratio is high. Thus, the only factor determining the actual hepatic clearance is the rate of supply of drug to the liver (or hepatic blood flow).

First pass liver clearance is important because even small changes in the extraction of drugs can cause large changes in bioavailability. For example, if the bioavailability of drug A by oral administration is 20% by the time it reaches the systemic circulation, and the same drug A by intravenous administration is 100%, absent no other complicating factors, the oral dose will therefore have to be 5 times the intravenous dose to achieve similar plasma concentrations.

Secondly, in some instances where liver enzyme activity is very high, drug formulations should be designed to have the drug pass directly through to the systemic circulation and avoid first pass liver clearance all together. For example, drugs administered intranasally, sublingual, buccal, rectal, vagina, etc. directly enter the systemic circulation and do not enter the hepatic portal blood circulation to be partially or fully extracted by the liver. Alternatively, where drugs cannot be administered by the above means, a tablet with at least one enteric-coating layer to prevent release of the drug in the stomach (i.e., highly acidic environment) is provided. Thus, an objective of the invention is to administer drugs using these alternative routes.

Additionally, first pass liver clearance is an important factor because many patients are on more than one drug regimen, and this may cause drug interactions which increase or decrease liver enzyme activity; thereby increasing or decreasing metabolism (increasing or decreasing the hepatic extraction ratio) of the drug of interest.

Hence, therapeutic compositions of the invention may be administered directly to the systemic circulatory system and avoid first pass liver clearance. Avoiding first pass clearance assures that more of the drug will be available to the system. Stated another way, by avoiding first pass liver clearance, the bioavailability of the drug is increased.

Embodiments of the present invention also relate to methods of increasing absorption of a low molecular weight CGRP inhibitor into the circulatory system of a subject including administering via the oral, ocular, nasal, nasolacrimal, inhalation, or the CSF delivery route the compound and an absorption increasing amount of a suitable non-toxic, non-ionic alkyl glycoside having a hydrophobic alkyl joined by a linkage to a hydrophilic carbohydrate.

The composition formulation is appropriately selected according to the administration route, such as oral administration (oral preparation), external administration (e.g., ointment), injection (preparations for injection), and mucosal administration (e.g., buccal and suppository) etc. For example, excipients (e.g., starch, lactose, crystalline cellulose, calcium lactate, magnesium aluminometasilicate and anhydrous silicate), disintegrators (e.g., carboxymethylcellulose and calcium carboxymethylcellulose), lubricants (e.g., magnesium stearate and talc), coating agents (e.g., hydroxyethylcellulose), and flavoring agents can be used for oral and mucosal formulations; whereas, solubilizers and auxiliary solubilizers capable of forming aqueous injections (e.g., distilled water for injection, physiological saline and propylene glycol), suspending agents (e.g., surfactant such as polysorbate 80), pH regulators (e.g., organic acid and metal salt thereof) and stabilizers are used for injections; and aqueous or oily solubilizers and auxiliary solubilizers (e.g., alcohols and fatty acid esters), tackifiers (e.g., carboxy vinyl polymer and polycarbohydrates) and emulsifiers (e.g., surfactant) are used for external agents. The CGRP inhibitor and the alkyl glycoside can be admixed, mixed, or blended along with the above excipients, disintegrators, coating polymers, solubilizers, suspending agents, etc., prior to administration, or they can be administered sequentially, in either order. It is preferred that they be mixed prior to administration.

As used herein, the term “mucosal delivery-enhancing agent” includes agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a compound(s) (e.g., biologically active compound). Enhancement of mucosal delivery can occur by any of a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms.

Exemplary mucosal delivery enhancing agents include the following agents and any combinations thereof:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from:
   (i) a surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. For example, EDTA or its salts (e.g., sodium or potassium) are employed in amounts ranging from about 0.01% to 2% by weight of the composition containing alkyl carbohydrate preservative.

Compounds whose absorption may be increased by the method described herein include any CGRP inhibitor now known or later discovered, in particular compounds that are difficult to administer by other methods, for example, compounds that are degraded in the gastrointestinal (GI) tract or that are not absorbed well from the GI tract, or compounds that subjects could administer to themselves more readily via the oral, ocular, nasal, nose-to-brain, nasolacrimal, inhalation or pulmonary, oral cavity (sublingual or Buccal cell), or CSF delivery route than by traditional self-administration methods such as injection.

As discussed herein, varying amounts of a CGRP inhibitor may be absorbed as the CGRP inhibitor passes through the buccal, sublingual, oropharyngeal and oesophageal pregastric portions of the alimentary canal. However, the bulk of the CGRP inhibitor passes into the stomach and is absorbed in the usual mode in which enteric dosage forms such as tablets, capsules, or liquids are absorbed. As the compound is absorbed from the intestines, the compound is brought directly into the liver, where, depending upon its specific chemical structure, it may be metabolized and eliminated by enzymes that perform the normal detoxifying processes in liver cells. This elimination is referred to as “first-pass” metabolism or the “first-pass” effect in the liver as previously discussed. The resulting metabolites, most often substantially or completely inactive compared to the original molecule, are often found circulating in the blood stream and subsequently eliminated in the urine and/or feces.

Aspects of the present invention are based on the finding that addition of certain alkyl carbohydrates, when included in fast-dispersing dosage forms, modulate the proportion of the CGRP inhibitor that is subject to the first-pass effect, thus allowing a fixed amount of the CGRP inhibitor to exert greater clinical benefit, or allowing a smaller amount of the CGRP inhibitor to achieve similar clinical benefit compared to an otherwise larger dose.

Thus, in an aspect of the invention the pharmaceutical compositions are prepared in oral solid molded fast-dispersing dosage form, such as described in U.S. Pat. No. 9,192,580, issued Nov. 24, 2015.

As used herein, the phrase “fast-dispersing dosage form” refers to compositions which disintegrate or disperse within 1 to 60 seconds, for example, 1 to 50 seconds, 1 to 40 seconds, 1 to 30 seconds, 1 to 20 seconds, 1 to 10 seconds, or 2 to 8 seconds, after being placed in contact with a fluid. The fluid is preferably that found in the oral cavity, i.e., saliva, as with oral administration.

In an embodiment, the compositions described herein are solid fast dispersing dosage forms including a solid network of the active ingredient, for example, zavegepant, and a water-soluble or water-dispersible carrier containing fish gelatin. Accordingly, the carrier is inert towards the active ingredient. The network is obtained by subliming solvent from a composition in the solid state, the composition including the active ingredient and a solution of the carrier in the solvent. The dosage forms according to the invention can be prepared according to the process disclosed in Gregory et al., U.K. Patent No. 1,548,022 using fish gelatin as the carrier. Accordingly, an initial composition (or admixture) including the active ingredient and a solution of the fish gelatin carrier in a solvent is prepared followed by sublimation. The sublimation is preferably carried out by freeze drying the composition. The composition can be contained in a mold during the freeze-drying process to produce a solid form in any desired shape. The mold can be cooled using liquid nitrogen or solid carbon dioxide in a preliminary step prior to the deposition of the composition therein. After freezing the mold and composition, they are next subjected to reduced pressure and, if desired, controlled application of heat to aid in sublimation of solvent. The reduced pressure applied in the process can be below about 4 mm Hg, preferably below about 0.3 mm Hg. The freeze dried compositions can then be removed from the mold if desired or stored therein until later use.

When the process is used with active ingredients and fish gelatin as the carrier, a solid fast-dispersing dosage form is produced having the advantages associated with the use of fish gelatin described herein. Generally, fish gelatin is categorized as being from cold water and warm water fish sources and as being of the gelling or non-gelling variety. The non-gelling variety of fish gelatin, in comparison to gelling fish gelatin and bovine gelatin, contains lower proline and hydroxyproline amino acid content, which are known to be associated with cross-linking properties and gelling ability. Non-gelling fish gelatin can remain at solution concentrations of up to about 40% as well as in temperatures as low as 20° C. In an aspect of the invention, the fish gelatin used in accordance with the invention is preferably obtained from cold water fish sources and is the non-gelling type of fish gelatin. More preferably, in an aspect of the invention, the non-hydrolyzed form of non-gelling fish gelatin is used. In an alternative embodiment, spray-dried non-hydrolyzed non-gelling fish gelatin may be used. Fish gelatins suitable for use in the compositions described herein are commercially available.

The compositions, according to embodiments of the invention, can also contain, in addition to the active ingredient arid fish gelatin carrier, other matrix forming agents and secondary components. Matrix forming agents suitable for use include materials derived from animal or vegetable proteins, such as other gelatins, dextrins and soy, wheat and psyllium seed proteins; gums such as acacia, guar, agar, and 10 xanthan; polycarbohydrates; alginates; carboxymethylcelluloses; carrageenans; dextrans; pectins; synthetic polymers such as polyvinylpyrrolidone; and polypeptide/protein or polycarbohydrate complexes such as gelatin-acacia complexes.

Other materials which may also be incorporated into the fast-dissolving compositions, according to embodiments of the present invention, include sugars such as mannitol, dextrose, lactose, galactose, and trehalose; cyclic sugars such as cyclodextrin; inorganic salts such as sodium phosphate, sodium chloride and aluminum silicates; and amino acids having from 2 to 12 carbon atoms such as glycine, L-alanine, L-aspartic acid, L-glutamic acid, L-hydroxyproline, L-isoleucine, L-leucine and L-phenylalanine. One or more matrix forming agents may be incorporated into the solution or suspension prior to solidification (freezing). The matrix forming agent may be present in addition to a surfactant or to the exclusion of a surfactant. In addition to forming the matrix, the matrix forming agent may aid in maintaining the dispersion of any active ingredient within the solution of suspension. This is especially helpful in the case of active agents that are not sufficiently soluble in water and must, therefore, be suspended rather than dissolved. Secondary components such as preservatives, antioxidants, surfactants, viscosity enhancers, coloring agents, flavoring agents, pH modifiers, sweeteners or taste-masking agents may also be incorporated into the fast-dissolving compositions. Suitable coloring agents include red, black and yellow iron oxides and FD & C dyes such as FD&C Blue No. 2 and FD&C Red No. 40 available from Ellis & Everard. Suitable flavoring agents include mint, raspberry, licorice, orange, lemon, grapefruit, caramel, vanilla, cherry and grape flavors and combinations of these. Suitable pH modifiers include the edible acids and bases, such as citric acid, tartaric acid, phosphoric acid, hydrochloric acid, maleic acid and sodium hydroxide. Suitable sweeteners include, for example, sucralose, aspartame, acesulfame K and thaumatin. Suitable taste-masking agents include, for example, sodium bicarbonate, ion exchange resins, cyclodextrin inclusion compounds, adsorbates or microencapsulated actives.

Increasing or decreasing the amount of specific alkyl carbohydrates included in fast-dispersing dosage forms may alter or modulate the site of absorption of the CGRP inhibitor, increasing or decreasing, respectively, that a proportion of the CGRP inhibitor that is absorbed through buccal tissue compared to other portions of the alimentary canal. In cases where it is desirable to speed the onset of drug action but preserve the normally longer Tₘₐₓ associated with the standard oral tablet, the alkyl glycoside content can be reduced to attenuate buccal absorption so that a portion of the drug is immediately absorbed buccally for rapid onset, but the rest is absorbed through the slower gastric absorption process. While not wishing to be bound by a theory, it is understood that by selecting an alkyl glycoside concentration less than, for example 20% less than, the concentration of alkylcarbohydrate that has been found by experiment to produce maximal or near maximal buccal absorption, a broader absorption peak in the “systemic drug level” versus time graph, overall, may be achieved where this is judged to be clinically desirable.

Further, in other aspects of the invention, addition of certain alkyl glycosides having specific alkyl chain lengths to the fast-dispersing tablets may alter the pharmacokinetics of pre-gastric drug absorption in beneficial ways. For example, incorporation of from between about 0.2%-0.3%, 0.3%-0.4%, 0.4%-0.5%, 0.5%-1.0%, 1.0%-2.0%, 2.0%-3.0%, 3.0%-4.0%, 4.0%-5.0%, 5.0%-6.0%, 6.0%-7.0%, 7.0%-8.0%, 9.0%-10.0%, and greater than 10% of alkyl glycoside may alter the pharmacokinetics of pre-gastric drug absorption in beneficial ways. In exemplary embodiments, the alkyl glycoside is dodecyl maltoside, tetradecyl maltoside and/or sucrose dodecanoate, which when incorporated into a fast-dispersing tablet format increases the drug that enters into systemic circulation and decreases the drug that is eliminated by the “first-pass” effect in the liver. Additionally, the time to maximum drug levels may be dramatically reduced, for example, from one to six hours, to approximately 15 to 45 minutes. For use in treating patients undergoing acute migraine episodes, this more rapid absorption of the CGRP inhibitor, resulting in more rapid onset of action, may be of great benefit.

Further, other aspects of the invention, when certain types of fast-dissolve or fast-dispersing tablets are placed between the cheek and gum or into close association with buccal tissue inside the mouth, an even larger proportion of the CGRP inhibitor is directly absorbed into systemic circulation and a smaller amount subsequently undergoes first pass elimination in the liver. Also, a particularly favorable location within the mouth for this effect is believed to be inside the central portion of the upper lip, between the inside of the lip and gums, directly below the nose. In exemplary aspects, these types of fast-dissolve dosage formulations are prepared by lyophilization or vacuum drying. In an exemplary aspect, the dosage formulation is prepared in a manner that results in a dosage formulation that is substantially porous.

As used herein, the term “fast-dispersing dosage form” is intended to encompass all the types of dosage forms capable of dissolving, entirely or in part, within the mouth. However, in exemplary aspects, the fast-dispersing dosage form is a solid, fast-dispersing network of the active ingredient and a water-soluble or water-dispersible carrier matrix, which is inert towards the active ingredient and excipients. As noted above, the network may be obtained by lyophilizing or subliming solvent from a composition in the solid state, which composition includes the active ingredient, an alkyl glycoside, and a solution of the carrier in a solvent. While a variety of solvents are known in the art as being suitable for this use, one solvent particularly well suited for use with the present invention is water. Water-alcohol mixtures may also be employed where solubility of the CGRP inhibitor in the mixed solvent is enhanced. For poorly water soluble drugs, dispersions of small drug particles can be suspended in an aqueous gel that maintains uniform distribution of the substantially insoluble drug during the lyophilization or subliming process.

In an embodiment, the dosage form may include the CGRP antagonist in an amount from about 10-80 weight %, for example, about 20-80 weight %, about 30-80 weight %, about 40-80 weight %, or about 50-80 weight % based on the total weight of the dosage form. The dosage form may further include the alkyl glycoside in an amount of about 0.01-50 weight %, for example, about 0.1-50 weight %, about 1-50 weight %, about 5-50 weight %, or about 10-50 weight % based on the total weight of the dosage form. The dosage form may further include fish gelatin in an amount of about 10-30 weight %, for example, about 15-30 weight % or about 20-30 weight % based on the total weight of the dosage form. The dosage form may further include about 10-25 weight % of a filler.

In an embodiment, the aqueous gel may be the self-assembling hydrogels described in U.S. Patent Application No. 60/957,960, formed using selected alkyl glycosides such as sucrose mono-and di-stearate and/or tetradecylmaltoside.

Matrix forming agents suitable for use in fast-dissolve formulations of the present invention are describe throughout this application. Such agents include materials derived from animal or vegetable proteins, such as the gelatins, collagens, dextrins and soy, wheat and psyllium seed proteins; gums such as acacia, guar, agar, and xanthan; polycarbohydrates; alginates; carrageenans; dextrans; carboxymethylcelluloses; pectins; synthetic polymers such as polyvinylpyrrolidone; and polypeptide/protein or polycarbohydrate complexes such as gelatin-acacia complexes. In exemplary aspects, gelatin, particularly fish gelatin or porcine gelatin is used.

While it is envisioned that virtually any CGRP antagonist may be incorporated into a fast-dissolve dosage formulation as described herein, particularly well suited CGRP antagonists have low oral bioavailability (for example, less than 80%), such as BHV-3500.

### ODT Formulations of Zavegepant

In an embodiment, the pharmaceutical composition may include a pharmaceutically acceptable carrier and a therapeutically effective amount of zavegepant, a solvate thereof, or a pharmaceutically acceptable salt thereof, wherein the pharmaceutical composition is in a form of an oral solid molded fast-dispersing dosage form. Such a pharmaceutical composition may be formulated, for example, as orally disintegrating tablet (ODT). An oral solid molded fast-dispersing dosage form of a CGRP inhibitor is described, for example, in International Application No. PCT/US2019/023940 filed on Mar. 25, 2019 and published as WO 2019/191008 A1 on Oct. 3, 2019, which is incorporated herein in its entirety by reference.

### Soft Gel Formulations of CGRP Inhibitors

In an embodiment, the soft gel pharmaceutical formulation may include:

a synthetic or natural poorly permeable calcitonin gene-related peptide (CGRP) inhibitor or salt or solvate thereof in an amount 0.01-20 weight % of the total weight of the formulation;

a lipophilic phase comprising triglycerides of fatty acids in an amount of 50-80 weight % of the total weight of the formulation; and

at least one lipophilic surfactant comprising partial esters of polyol and fatty acids in an amount of 10-50 weight % of the total weight of the formulation.

The above formulation is described, for example, in International Application No. PCT/US2020/027800 filed on Apr. 10, 2020 and published as WO 2020/210722 A1 on Oct. 15, 2020, which is incorporated herein in its entirety by reference.

### Methods of Treatment

An embodiment provides a method for treatment or prevention of a condition associated with aberrant levels of CGRP in a subject in need thereof, wherein the method comprises administering to the subject any of the above pharmaceutical compositions and formulations.

The CGRP inhibitor may be a CGRP antibody, a CGRP receptor antibody, an antigen-binding fragment from a CGRP antibody or a CGRP receptor antibody, a CGRP infusion inhibitory protein, a CGRP bio-neutralizing agent, a small molecule CGRP receptor antagonist, a small molecule CGRP inhibitor, or a polypeptide CGRP inhibitor. The small molecule CGRP receptor antagonist is zavegepant, rimegepant, ubrogepant, atogepant, telcagepant, or olcegepant, a solvate thereof, or a pharmaceutically acceptable salt thereof.

In an embodiment, the condition may be a disorder selected from acute migraine, chronic migraine, cluster headache, chronic tension type headache, medication overuse headache, post-traumatic headache, post-concussion syndrome, brain trauma, and vertigo.

In another embodiment, the condition may be a disorder selected from chronic pain, neurogenic vasodilation, neurogenic inflammation, inflammatory pain, neuropathic pain, diabetic peripheral neuropathic pain, small fiber neuropathic pain, Morton's neuroma, chronic knee pain, chronic back pain, chronic hip pain, chronic finger pain, exercise-induced muscle pain, cancer pain, chronic inflammatory skin pain, pain from burns, pain from scars, complex regional pain syndrome, burning mouth syndrome, alcoholic polyneuropathy, chronic inflammatory demyelinating polyradiculoneuropathy, human immunodeficiency virus (HIV) or acquired immunodeficiency syndrome (AIDS)-associated neuropathy, drug-induced neuropathy, industrial neuropathy, lymphomatous neuropathy, myelomatous neuropathy, multi-focal motor neuropathy, chronic idiopathic sensory neuropathy, carcinomatous, neuropathy, acute pain autonomic neuropathy, compressive neuropathy, vasculitic/ischaemic neuropathy, tempero-mandibular joint pain, post-herpetic neuralgia, trigeminal neuralgia, chronic regional pain syndrome, eye pain, and tooth pain.

In an example, the condition may be medication overuse headache (MOH), and the subject having the condition may be undergoing treatment for pain, wherein the treatment for pain may include a medicament selected from acute pain medications and chronic pain medications. For example, the treatment for pain includes a medicament selected from triptans, ergot alkaloids, analgesics and opioids. The triptans may be selected from rizatriptan, sumatriptan, naratriptan, eletriptan, donitriptan, almotriptan, frovatriptan, avitriptan, and zolmitriptan. The ergot alkaloids may be selected from clavines, lysergic acid amides and ergopeptines. The ergot alkaloid may also be selected from ergonovine, methylergonovine, methysergide, ergotamine, dihydroergotamine, bromocriptine, ergoloid mesylates and lysergic acid diethylamide, or a combination thereof.

The MOH may result from the chronic use of one or more pain medications. The subject may have a primary headache disorder selected from migraine, cluster-type headache, or tension-type headache. The subject may be currently undergoing treatment or may have received treatment for the primary headache disorder.

The treatment for pain may include a medicament selected from aspirin, diclofenac; diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib, etoricoxib, valdecoxib, parecoxib, meloxicam, lumiracoxib, or a combination thereof.

The MOH may result from treatment with a medicament selected from ketamine, esketamine, alfentanil, alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, and zolpidem.

The MOH may result from the chronic use of a medicament selected from alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, and zolpidem.

The MOH may result from the chronic use of a medicament selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanyl, tramadol, prochlorperazine, celecoxib, rofecoxib, meloxicam, piroxicam, JTE-522, L-745,337, NS388, deracoxib, valdecoxib, iumiracoxib, etoricoxib, parecoxib, 4-(4-cyclohexyl-2-methyloxazol-5-yl)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl) phenyl]-3(2H) pyridazinone, 2-[(2,4-dichloro-6-methylphenyl) amino]-5-ethyl-benzeneacetic acid, (3Z) 3-[(4-chlorophenyl) [4-(methylsulfonyl)phenyl] methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, ciltiazem, Nifedipine, lidocaine, tetracaine, prilocaine, bupivicaine, mepivacaine, etidocaine, procaine, benzocaine, phehelzine, isocarboxazid, dichloralphenazone, nimopidine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, haloperidol, metoclopramide, nabilone, thiethylperazine, trimethobenzemide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

In another example, the condition may be post-traumatic headache (PTH) headache, and the subject having the condition may experience a PTH one, two, three, four, five, six or seven days after a traumatic incident. The traumatic incident may result in a concussion or loss of consciousness. The subject may suffers from dizziness, insomnia, poor concentration, memory problems, photophobia, phonophobia, or fatigue, or a combination thereof.

In another embodiment, the condition may be a disorder selected from non-insulin dependent diabetes mellitus, vascular disorders, inflammation, arthritis, thermal injury, circulatory shock, sepsis, alcohol withdrawal syndrome, opiate withdrawal syndrome, morphine tolerance, hot flashes in men and women, flushing associated with menopause, allergic dermatitis, psoriasis, encephalitis, ischaemia, stroke, epilepsy, neuroinflammatory disorders, neurodegenerative diseases, skin diseases, neurogenic cutaneous redness, skin rosaceousness, erythema, tinnitus, obesity, inflammatory bowel disease, irritable bowel syndrome, vulvodynia, polycystic ovarian syndrome, uterine fibroids, neurofibromatosis, hepatic fibrosis, renal fibrosis, focal segmental glomerulosclerosis, glomerulonephritis, IgA nephropathy, multiple myeloma, myasthenia gravis, Sjogren's syndrome, osteoarthritis, osteoarthritic degenerative disc disease, temporomandibular joint disorder, whiplash injury, rheumatoid arthritis, and interstitial cystitis. The method according to claim 61, wherein the skin disease are selected from recurrent herpes, contact hypersensitivity, prurigo nodularis, chronic pruritus, and uremic pruritus.

In another embodiment, the condition may be a disorder selected from chronic obstructive pulmonary disease, pulmonary fibrosis, bronchial hyperreactivity, asthma, cystic fibrosis, chronic idiopathic cough, and a toxic injury. The toxic injury is selected from chlorine gas injury, mustard gas injury, acrolein injury, smoke injury, ozone injury, warfare chemical exposure, and industrial chemical exposure.

The present invention is more particularly described in the following examples which are intended as illustrative only since numerous modifications and variations therein will be apparent to those skilled in the art. The following examples are intended to illustrate but not limit the invention.

### EXAMPLES

### Example 1

In the present example, the pharmacokinetics (PK) of BHV-3500 after a single sublingual (tablet) dose of BHV-3500 in dogs were investigated and PK parameters were determined.

BHV-3500 (zavegepant) is a high affinity (human CGRP Kᵢ=0.023 nM), selective and structurally unique small molecule CGRP receptor antagonist having the following formula I:

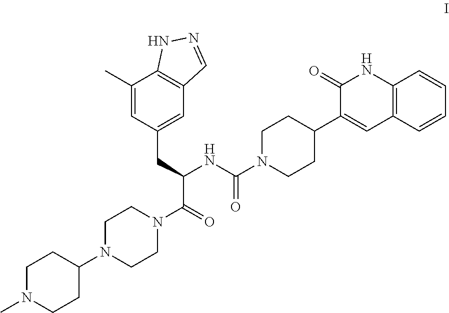 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

The chemical name of BHV-3500 is (R)-N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide. BHV-3500 is described, for example, in WO 03/104236 published Dec. 18, 2003 and U.S. Pat. No. 8,481,546 issued Jul. 9, 2013, which are incorporated herein in their entireties by reference. BHV-3500 has poor permeability and was selected as an object of the present study. BHV-3500-d8, which is an octadeuterated analog of BHV-3500 has the following formula II:

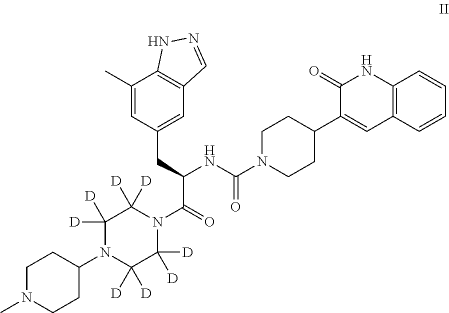 [SMILES: [2H]C1([2H])N(C(=O)[C@@H](Cc2cc(C)c3[nH]ncc3c2)NC(=O)N2CCC(c3cc4ccccc4[nH]c3=O)CC2)C([2H])([2H])C([2H])([2H])N(C2CCN(C)CC2)C1([2H])[2H]]

### Description of Study Protocol

Study Title: Single dose sublingual PK study of BHV-3500 in dogs.

Study Objective: To determine the pharmacokinetics of BHV-3500 tablet formulations after a single sublingual dose in dogs.

Duration of Study: 3 weeks.

### Test Article Formulation

Identification. The test article is identified as BHV-3500. The test article will be supplied as tablets.
Hazard to Personnel. Routine safety procedures used for handling of hazardous or potentially hazardous chemicals will be followed to ensure the health and safety of personnel handling the test article.
Test Article Characterization. A certificate of analysis (or other appropriate documentation) verifying the identity or purity of test articles will be provided.
Dose Preparation and Analysis. No analysis will be performed on the dosing formulations.
Storage. The BHV-3500 tablets will be stored at room temperature.
Sample Disposition and Retention. All quantities of the test articles that are dispensed will be documented. Retention samples are not required for a study of this duration.
Basis for Selection of Doses of Test Articles. The test articles dose levels were selected on the basis of previous PK studies with the test articles.
Route of Administration. The test article will be administered sublingually.

Disposition of Test Article. Upon completion of the study, any remaining test articles will be returned and discarded.

### Experimental Design

See Table 1 below.

### Test System

Test Animals. Three (3) or 3 female beagle dogs are obtained from Ridglan Farms, Mount Horeb, WI for use in this study. All animals are immunized against distemper, type 2 adenovirus, parainfluenza, Bordetella, rabies, papilloma virus, and parvovirus by the supplier. Dogs will be approximately one year old and weigh approximately 8 to 12 kg at the initiation of dosing. The same 3 animals will be used for all test article administrations.
Justification. The dog is a standard species used for non-clinical toxicity studies, and is accepted by the U.S. Food and Drug Administration as a large animal (non-rodent) model system for the safety assessment of pharmacokinetics of pharmaceutical agents.
Justification for Number of Animals. The number of animals used is the minimum necessary to obtain meaningful data. To the knowledge of the Sponsor and the Study Director, conduct of this study will result in no unnecessary duplication of existing data with regard to species, test article, does(s), route, and duration of administration.
Housing. Dogs will be housed individually in pens equipped with automatic watering systems. Pens will be cleaned daily. Dogs will be housed in accordance with U.S. Department of Agriculture Welfare Standards (Title 9, Code of Federal Regulation, Part 3, 1991 Revision) and standards set forth in the Guide for the Care and Use of Laboratory Animals (National Research Council, 2011).
Food. Certified Canine Diet #2021C (Harlan Teklad, Madison, WI). Approximately 400 g of food will be made available to each dog daily for a minimum of 2 hours. Each lot of diet is analyzed for contaminants to ensure that none are present at concentrations which would be expected to interfere with the conduct or purpose of this study. Analytical data from the lots of diet to be used in the study will be retained on file at the testing facility. Dogs will be fasted prior to dosing. Food will be provided approximately one hour after dosing.
Water. Coarse-filtered City of Chicago water will be provided ad libitum to all dogs via an automatic watering system. Water is analyzed periodically for bacterial contamination and chemical composition (e.g., electrolytes, metals, etc.). Water analysis records are retained on file at the testing facility. No contaminants expected to interfere with the study are known to be present in the water.
Animal Identification. Each dog will be identified by a USDA tattoo number in the right or left ear. Each dog will also be assigned a unique number within the study. All pens will be identified by the Project Number, Animal Number, and Sex. Cage cards will be color-coded according to group.
Environmental Control. Temperature and relative humidity in the animal room will be recorded manually each day. A 12-hour light/dark cycle (maintained with an automatic timer) will be used. Animal rooms will be held within temperature and relative humidity ranges of approximately 20° C. to 25° C. and 30%-70%, respectively.

### Methods

Quarantine. Animals purchased for this study will be held in quarantine for at least two weeks prior to administration of the test article. Throughout the quarantine period, animals, will be observed at least once daily for mortality or evidence of moribundity.
Randomization. After animals have been released from quarantine, animals will be randomly assigned into groups. Prior to randomization, each dog will receive a detailed clinical observation to ensure its suitability as a test animal.
Administration. Animals in groups 1 to 2 will receive a single oral capsule administration of BHV-3500 at 20 mg/dog. Animals in groups 4 to 6 will receive a single oral (capsule) administration of BHV-3500 at a dose of 50 mg/dog. Each group will be followed by a washout period of at least 48 hours prior to the next group being dosed.
Moribundity/Mortality Observations. Prior to initiation of dosing, animals will be observed at least once daily for mortality or evidence of moribundity. Upon initiation of dosing and then throughout the remainder of the observation periods, all surviving study animals will be observed at least twice daily for mortality or evidence of moribundity and to assess their general health. Any abnormal clinical signs will be recorded. Moribundity/mortality checks will be separated by a minimum of four hours.
Moribund Animals. During the moribundity/mortality observations, any animal judged not likely to survive until the next scheduled observation period will, upon consent of the Attending Veterinarian and Study Director, be removed from the study, weighed, euthanized, and necropsied. These animals will be recorded in the study notebook as being euthanized in extremis. Dead animals will be immediately removed for necropsy and the death will be recorded in the study notebook.
Injured or Diseased Animals. Animals on test will be treated for any disease or injury in conformance with standard veterinary practice. A complete record of the circumstances and the disposition of any affected animals will be made in the study notebook. Any animal that pose a potential infectious threat to other studies will be isolated.
Clinical Observations. Clinical observation will be done approximately 1 hour after each dose administration.
Body Weight Management. Animals will be weighed prior to each dose.
Food Consumption Measurements. Individual animal food consumption will not be measured in this study.
Plasma Drug Level. Blood samples (approximately 3 mL collected from the jugular vein) for determination of plasma levels of BHV-3500 will be obtained from each dog at six time points (pre-dose; 15, 30, and 60 minutes, 2 and 4 hours) after each dose. EDTA will be used as the anticoagulant. Plasma samples will be frozen at approximately −70° C. until analyzed at the testing center for concentration of BHV-3500. Pharmacokinetic modeling will include AUC, t_{1/2}, Tₘₐₓ, and Cₘₐₓ.
Postmortem. This is a non-terminal study. The dogs will returned to quarantine after the last blood collection.
Data Notebooks. All original paper data generated by the testing center will be maintained in loose-leaf notebooks. Paper data to be maintained in loose-leaf notebooks will include, but not necessarily be limited to, the following:

the original signed Protocol and any amendments and/or deviations;

animal receipt records;

animal care records;

test article data;

blood collection data;

TK data

Data captured electronically using ToxData® (e.g., dose administration, daily moribundity/mortality and environmental data, clinical observations, body weights, etc.) will be maintained within the computer system's database; electronic copies of the ToxData®.htm files will also be backed-up onto CD-ROM(s) and the disc(s) will be maintained with the raw data.
Alteration of Design. Alterations in the Protocol may be as the study progresses in the form of a protocol amendment. No changes in the Protocol will be made without the specific written consent of the Sponsor.

sRegulatory Standards and Compliance. Due to the pilot nature of this study, the study will not be conducted in compliance with Good Laboratory Practice (GLP) regulations set forth by the U.S. FDA (Title 21 of the Code of Federal Regulations, Part 38). The study will be conducted in accordance with the test center standard operating procedures.

Report. A draft version of the report will be prepared and submitted to the Sponsor for review. Information in the report will include, but not necessarily be limited to, the following:

Copy of the approved protocol, including any amendment and/or deviations

Species and strain of animal used

Clinical observation data

Body weight data

Plasma drug level data

Pharmacokinetic data

Following Sponsor review of the draft report, a final report will be submitted to the Sponsor.
Data Retention. All raw data generated as a result of this study and a copy of the final report from the study will be archived at the testing center for a period of one year from the date of completion of the study. The Sponsor will be responsible for all costs associated with continued storage of the archival materials in the testing center archives or for the shipment of these materials to another storage facility. The testing center QAU will maintain a complete record of the disposition of all archival materials.
Personnel. Curricula vitae for all testing center personnel involved in the execution of the study are on file at the testing center.
Protocol Approval. This protocol complies with the specific documents of the Sponsor.

### Methods

### 1. Experimental Design and Dose Administration

For each group, three female beagle dogs were dosed once with BHV-3500 or FC-10475 as detailed in Table 1:

TABLE 1
Experimental Design and Dose Administration

| Group | Test Article | Route | Vehicle | Dose |
|---|---|---|---|---|
| 1 | BHV-3500 | Sublingual | Z4840/108/3a | 50 mg |
| | | (tablet) | [HMW Fish | |
| | | | Gelatin | |
| | | | (5.00% w/w), | |
| | | | Mannitol | |
| | | | (4.00% w/w), | |
| | | | BHV-3500 (8.80% | |
| | | | w/w), DDM | |
| | | | (0.25% w/w), Purified | |
| | | | Water (81.95% w/w)] | |
| 2 | BHV-3500 | Sublingual | Z4840/108/3a without | 50 mg |
| | | (tablet) | DDM | |

### 2. Blood Collection

After each dose, blood samples (approximately 3 mL from the jugular vein) for determination of plasma levels of BHV-3500 were obtained from of each dog at six time points (pre-dose; 15, 30, and 60 minutes and 2 and 4 hours post-dose). EDTA was used as the anticoagulant. Plasma samples were frozen at approximately −70° C. until analyzed.

### 3. Reference and Internal Standards and Plasma Sample Preparation

The reference standards for BHV-3500 and BHV-3500-d8 were provided by the Sponsor and stored at room temperature. The standards were used without further purification for the preparation of calibration standards and quality control (QC) samples for the determination of BHV-3500 concentrations in plasma samples collected during this study.

For the determination of BHV-3500 in plasma, a 50 μL aliquot from each sample was transferred into the appropriate well of a 96-well plate to which 10 μL of 50% acetonitrile (ACN) in water was added, followed by 250 μL of internal standard solution (10 ng/mL BHV-3500-d8 in ACN). After sealing the plate and vortexing for approximately 5 minutes, the plate was centrifuged at 4000 rpm for 10 minutes at 4±4° C. A portion (100 μL) of the resulting supernatant was transferred to into the appropriate well (containing 300 μL 0.15% formic acid in water) of another 96-well plate. This plate was sealed and its contents mixed prior to instrumental analysis.

Freshly prepared BHV-3500 standard curves and QC samples were analyzed along with the study samples. Instrument calibrators were prepared by adding 10 μL of a stock BHV-3500/FC-10475 solution to 50 μL of blank dog plasma. Blank dog plasma was sourced from BioIVT (Hicksville, NY) and stored frozen at −20° C. Nominal calibrator concentrations ranged from 2.00 to 200 ng/mL. QC samples were prepared at concentrations of 6.00, 50.0, and 150 ng/mL. Calibrators and QC samples were processed for analysis following the extraction procedure described above.

### 4. Analytical Equipment and Conditions

Calibrator, QC and study samples were analyzed under LC-MS/MS instrument conditions detailed in Table 2. Calibration curves were calculated from the linear regression (weighting factor of 1/x²) of the analyte to internal standard peak area ratios versus the analyte concentrations. Concentrations of analyte in the samples were determined using the peak area ratios and the regression parameters of the calibration curves.

TABLE 2
Instrument Operating Conditions

| | |
|---|---|
| SYSTEM: | Triple Quad 5500 LC-MS-MS (SCIEX; Framingham, MA) |
| | equipped with a Agilent 1100 Series LC System (Agilent |
| | Technologies, Wilmington, DE) |

HPLC CONDITIONS

| HPLC Column: | Kinetex C18; 50 × 2.1 mm; 5 μm (Phenomenex, Torrance, CA) | | |
|---|---|---|---|
| Column Temperature | 25° C. | | |
| Injection Volume: | 5 μL | | |
| Flow Rate: | 400 μL/min | | |
| Mobile Phase A: | 0.1% formic acid in water | | |
| Mobile Phase B: | 0.1% formic acid in acetonitrile | | |
| | Time | Mobile | Mobile |
| Program: | (minutes) | Phase A (%) | Phase B (%) |
| | 0.00 | 80 | 20 |
| | 0.30 | 80 | 20 |
| | 1.50 | 40 | 60 |
| | 3.50 | 40 | 60 |
| | 4.00 | 80 | 20 |
| | 7.00 | 80 | 20 |

| | |
|---|---|
| Run Time: | 7 minutes |
| Retention Time: | BHV-3500 and BHV-3500-d8: approximately 0.9 minutes |
| | FC-10475: approximately 2.6 minutes |

MS-MS CONDITIONS

| | |
|---|---|
| Scan Type: | MRM |
| Ion Source: | Turbo Spray ESI |
| Polarity: | Positive |
| Ion Source Temperature: | 500° C. |
| Ion spray Voltage: | 5000 Volts |
| Collision Energy: | BHV-3500 and BHV-3500-d8: 26 Volts |
| | FC-10475: 40 Volts |
| Ions monitored (Q1→Q3): | BHV-3500: 639.4→456.3 BHV- |
| | 3500-d8: 647.4→456.3 FC- |
| | 10475: 855.4→672.3 |
| Resolution: | Unit |
| Data System: | Analyst ® 1.6.3 (SCIEX; Framingham, MA) |

### 5. Pharmacokinetics

Individual animal plasma BHV-3500 concentration data at scheduled (nominal) sampling times were analyzed using the non-compartmental model for extravascular administration with Phoenix WinNonlin software (Version 8.1; Certara, Princeton, NJ).

Elimination rate constant values (λ_{z}) were calculated by log-linear regression on data points of the terminal phase (using Phoenix WinNonlin's Best Fit Lambda Z Calculation Method option) when allowed by the data; the plasma elimination half-life (t_{1/2}) was calculated as In(2)/λ_{z}. Area under the plasma concentration-time curve values from time zero to the concentration at the 4 hour time point (AUC₀₋₄ₕᵣ) were calculated by the linear-up/log-down trapezoidal rule.

Nominal dose levels were used for PK analysis. The PK parameters listed below were evaluated (as applicable and when allowed by the data).

Elimination half-life (t_{1/2})

Time of occurrence of maximum plasma concentration (Tₘₐₓ)

Maximum plasma concentration (Cₘₐₓ)

Area under plasma concentration-time curve [0 to the 4 hour time point; AUC₀₋₄ₕᵣ]

PK abbreviations and units of measure are presented in Table 3.

TABLE 3
PK Parameter Definitions and Abbreviations

| Parameter | Unit | Definition |
|---|---|---|
| R_{sq} | N/A | Correlation of the line |
| | | fitting the terminal phase |
| t_{1/2} | hr | Elimination half-life, |
| | | determined by ln(2)/λ_{Z} |
| Tₘₐₓ | hr | Time of occurrence of |
| | | maximum plasma |
| | | concentration |
| Cₘₐₓ | ng/mL | Maximum observed plasma |
| | | drug concentration |
| AUC_{0-4 hr} | hr*ng/mL | Area under the plasma |
| | | concentration-time curve |
| | | from time zero to the 4 |
| | | hour time point |

### Results

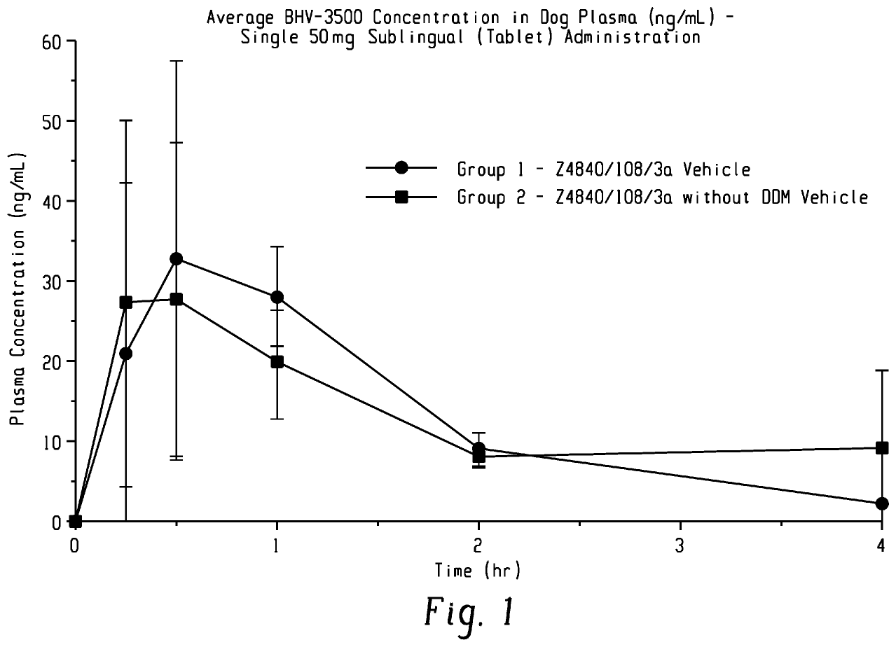

BHV-3500 concentration determinations are presented in Table 4, and are shown graphically in FIG. 1. PK parameters are presented in Table 5 (BHV-3500).

TABLE 4
BHV-3500 Concentration in Dog Plasma

| | | Blood Collection Time Point | | | | | |
|---|---|---|---|---|---|---|---|
| | | | Hours Post-Dose | | | | |
| | Female | Pre-Dose | 0.25 | 0.5 | 1 | 2 | 4 |
| Group | Animal ID | BHV-3500 Concentration (ng/ml) | | | | | |
| Group 1 | 01-PWS | BQL | 7.03 | 17.5 | 22.1 | 6.57 | BQL |
| Sublingual (Tablet) | 05-GJU | BQL | 45.6 | 61.2 | 34.4 | 10.2 | 2.15 |
| 50 mg BHV-3500 in a | 06-VWU | BQL | 9.54 | 19.2 | 27.6 | 10.2 | 2.33 |
| vehicle of | Average: | BQL | 20.7 | 32.6 | 28.0 | 8.99 | 2.24 |
| Z4840/108/3a | STD: | — | 22 | 25 | 6.2 | 2.1 | 0.13 |
| | % RSD: | — | 104 | 76 | 22 | 23 | 5.7 |
| Group 2 | 01-PWS | BQL | 5.34 | 7.96 | 12.0 | 7.82 | 16.1 |
| Sublingual (Tablet) | 05-GJU | BQL | 51.0 | 47.1 | 25.9 | 6.91 | BQL |
| 50 mg BHV-3500 in a | 06-VWU | 3.33 | 25.1 | 28.1 | 21.9 | 9.42 | 2.06 |
| vehicle of | Average: | 3.33 | 27.1 | 27.7 | 19.9 | 8.05 | 9.08 |
| Z4840/108/3a | STD: | — | 23 | 20 | 7.2 | 1.3 | 9.9 |
| without DDM | % RSD: | — | 84 | 71 | 36 | 16 | 109 |

TABLE 5
BHV-3500 PK Parameter Analysis Results

| | | PK Parameter | | | | |
|---|---|---|---|---|---|---|
| | Female | | t_{1/2} | Tₘₐₓ | Cₘₐₓ | AUC_{0-4 hr} |
| Group | Animal ID | R_{sq} | (hr) | (hr) | (ng/mL) | (hr*ng/mL) |
| Group 1 | 01-PWS | NC | NC | 1 | 22.1 | NC |
| Sublingual (Tablet) | 05-GJU | 0.9859 | 0.77 | 0.5 | 61.2 | 66.9 |
| 50 mg BHV-3500 in a | 06-VWU | NC | NC | 1 | 27.6 | 43.4 |
| vehicle of Z4840/108/3a | Median/Average: | — | 0.77 | 1 | 37.0 | 55.1 |
| | STD: | — | — | — | 21 | 17 |
| | % RSD: | — | — | — | 57 | 30 |
| Group 2 | 01-PWS | NC | NC | 4 | 16.1 | 40.3 |
| Sublingual (Tablet) | 05-GJU | 0.9994 | 0.54 | 0.25 | 51.0 | 49.3 |
| 50 mg BHV-3500 in a | 06-VWU | 0.9993 | 0.88 | 0.5 | 28.1 | 43.6 |
| vehicle of Z4840/108/3a | Median/Average: | — | 0.71 | 0.5 | 31.7 | 44.4 |
| without DDM | STD: | — | — | — | 18 | 5 |
| | % RSD: | — | — | — | 56 | 10 |

Group 1 where BHV-3500 was administered with DDM had Cₘₐₓ 37.0 ng/mL versus 31.7 ng/mL where BHV-3500 was administered without DDM (a 16% increase in Cₘₐₓ).

Group 1 where BHV-3500 was administered with DDM had AUC 55.1 hr*ng/mL versus 44.4 h*ng/mL where BHV-3500 was administered without DDM (a 24% increase in AUC).

### Example 2

The purpose of the study described in this example was to evaluate the technical feasibility of formulating BHV-3500 into the Zydis® dosage form. The feasibility determined the maximum dose strengths to be 50 mg free base, corresponding to 52.85 mg of the Hydrochloride salt (salt equivalency factor: 1.057).

The formulation feasibility activities consisted of five manufacturing studies in an attempt to develop a robust formulation containing BHV-3500 and Dodecyl Maltoside, with acceptable critical quality attributes, such as satisfactory finished product appearance and dispersion time.

### Study 1

Study 1 consisted of 5 batches. All batches contained a fixed concentration of Gelatin, mannitol and Dodecyl Maltoside. Study 1 investigated:
Determination of the maximum achievable dose of BHV-3500 that con be formulated into a Zydis® unit.
Assess four wet fill weights—250 mg/500 mg and 300 mg/600 mg (dose proportional formulations) and five concentration of BHV-3500 (% w/w).
Assessment of Dodecyl Maltoside (0.50% w/w) as a permeation enhancer to aid API absorption.
pH altered to pH 5.0±0.2.

Overall finished product appearance for the majority of batches was acceptable, a higher than anticipated dose of BHV-3500 (17.62% w/w) was frozen and freeze dried successfully. Extensive feathering was present in all batches, batches with a wet fill weight (500 mg/600 mg) had a higher level of feathering.

Popping out was also identified as a problem, particularly in units with a higher concentration of BHV-3500 and larger wet fill weight (batches Z4840/94/5a, 5b-16.67% w/w API at 300 mg/600 mg wet fill weight respectively).

All batches had dispersion times within the acceptable criterion of seconds.

It was recommended optimization of matrix formers such as gelatin and mannitol concentrations is required to improve finished product appearance, robustness of the units and reduce the feathering/friability and popping out. Also ranging of the permeation enhancer dodecyl Maltoside should be conducted to identify its effect on the overall finished product appearance of the units.

### Study 2

The purpose of Study 2 was to optimize formulation containing 16.67% w/w BHV-3500 to improve the physical appearance and rigidity. Study 2 investigated:
Assess the incorporation of different levels of the permeation enhancer dodecyl maltoside (0.2-0.5% w/w).
Assess two different fill weights 300 mg/600 mg (dose proportional formulations).
Assess HMW fish gelatin concentrations (5.50-6.00% w/w) and mannitol (4.40-4.80% w/w)
pH altered to pH 5.0±0.2.

All batches manufactured had very high incidences of cracking and popping out, due to this no dispersion testing was conducted. Based on the poor quality of all the batches manufactured, further formulation optimization was required to improve product appearance and quality. Increased levels of Gelatin and Mannitol did not improve appearance. Therefore, Glycine was to be used as a potential structure enhancer. DDM concentrations used as a potential permeation enhancer also needed to be optimized in the next study. It was also recommended to not alter the pH in future studies in order to determine the effect of citric acid on the finished product appearance.

### Study 3

The purpose of study 3 was to optimize the formulation containing 16.67% w/w BHV-3500 and 0.50% w/w DDM to improve the physical appearance and rigidity. Study 3 investigated:
Assessment of the incorporation of glycine (1.50% w/w) as a structural enhancer.
Assess one wet fill weight—600 mg.
No pH adjustment.

Overall all batches manufactured in Study 3 showed varying levels of cracking, and it is clear the presence of Glycine did not aid with the reduction of cracking. No improvement was seen in batches with or without DDM.

All units manufactured in this study popped out of their pockets upon inverting the tray. Furthermore, dispersion times for units containing both DDM and Glycine (batch Z4840/104/1) were outside the acceptable criterion of ≤10 seconds and the appearance of this batch was the poorest out of all batches; this suggested that the combination of DDM and Glycine should not be taken forward into future studies.

The oven study showed that between day 0 and day 14, there was no change in assay or related substances over the time period. There was sharp change between day 14 and day 21—this was because of an inadvertent temperature excursion where the temperature of the oven was increased to 105° C. After the oven was turned down to 50° C., there was no change in assay or related substances.

It was recommended that the next feasibility study should aim to range the % w/w of DDM and gelatin in order to aid with the cracking, and to also reduce the dosage of BHV-3500 to assess the impact on finished product appearance. It was recommended that this is performed as part of an experimental design.

### Study 4

The purpose of study 4 was to range the HMW fish gelatin whilst maintaining the ratio of mannitol, as well as ranging the concentration of DDM, and also to assess the effects of fill weight and solution hold time of the finished product appearance as part of an experimental design study. A larger pocket size was also to be used to lower the concentration of BHV-3500. Study 4 investigated:
% w/w HWM fish gelatin
% w/w HWM DDM
No pH adjustment
50 mg/100 mg dose in 600 mg/1200 mg pocket

This Study showed that an increase in dose and unit size from 50 mg dose/600 mg pocket size to 100 mg dose/1200 mg pocket size had a significant effect on the overall appearance and dispersion of the units at the same API concentration. The 100 mg dose/1200 mg pocket size units also displayed an increase in popping out and cracking. DDM appeared to have a negative effect of the dispersion times of the units at the higher concentration of 0.5% w/w. Therefore, the reduced concentration of 0.25% DDM (w/w) and a 600 mg fill weight was taken forward for further formulation optimization studies.

### Study 5

The purpose of study 5 was to manufacture the successful batch from study 4 containing 0.25% w/w DDM and 5.00% w/w HMW fish gelatin to provide samples for an informal stability study. Also to produce units not containing DDM to compare the impact of DDM on the finished product appearance. Study 5 investigated:
Assessment of the impact of Dodecyl Maltoside 0.25% w/w.

All batches were successfully incorporated into the Zydis® matrix, producing white circular units, with good appearance and dispersion. Both mixes for batches Z4840/120/1-2 were reported to become cloudy when left stirring over a 24-hour period, however upon review of images it was determined that the appearance of the solutions did not change over a 24 hour hold period and so were stable. This was also confirmed by assay and related substance testing which demonstrates units at 24 solution hold had the same assay results and no difference in the related substances or quantities found at both time points, informal stability ongoing.

Batch Z4840/120/1 containing 0.25% w/w of DDM did not have any significant defects when compared to batch Z4840/120/2 which did not contain DDM. This therefore suggested DDM does not significantly affect the overall finished product appearance at this concentration.

Batch Z4840/120/1 did however have air bubbles present in the units whereas batch Z4840/120/2 did not, this could suggest the presence of DDM causes air bubbles during dosing, which could be due to an increase in viscosity when DDM is present in the mix. However, this was not considered to be a significant defect due to the nature of the small batch size and hand dosing, this will be optimized for larger scale batches in the future.

### Formulation and Design Space

Details of the formulation evaluated during these studies are provided in Table 6. Ranging was performed for API concentration, gelatin and mannitol levels, dose weight and permeation enhancer (Dodecyl Maltopyranoside). Further evaluation of formulation and process parameters is recommended during formulation optimization.

TABLE 6
Formulation Design Space for Zydis ® BHV-3500 50 mg (600 mg wet fill weight)

| | | Control space 1 | Design Space | Knowledge Space | | Critical |
|---|---|---|---|---|---|---|
| Material | Function | (% w/w) | (% w/w) | (% w/w) | CQA | Material |
| Gelatin | Matrix former | 5.00 | 4.00-5.00 | 4.00-6.00 | Appearance, assay, | Yes |
| EP/USP/JP | | | | | dose uniformity, | |
| (Fish HMW) | | | | | disintegration/ | |
| | | | | | dispersion | |
| Mannitol | Matrix former | 4.00 | 3.20-4.00 | 3.20-4.80 | Appearance, | Yes |
| EP/USP/JP | | | | | disintegration/ | |
| | | | | | dispersion | |
| BHV-3500 | API | 8.80 | 6.00-8.80 | 6.00-16.67* | Appearance, assay, | Yes |
| | | | | | dose uniformity, | |
| | | | | | disintegration/ | |
| | | | | | dispersion, degradation | |
| | | | | | products | |
| Dodecyl | Permeation | 0.25 | 0.20-0.50 | 0.20-0.50 | Appearance, | Yes |
| Maltoside | Enhancer | | | | Permeation | |
| | | | | | enhancement, | |
| | | | | | disintegration/ | |
| | | | | | dispersion | |
| Glycine | Structural | N/A | N/A | 1.50 | Appearance, | No |
| | Enhancer | | | | disintegration/ | |
| | | | | | dispersion. | |
| Citric Acid | pH Modifier | N/A | N/A | 0.09-2.58 | Appearance, | No |
| | | | | | dispersion/dispersion. | |

*Salt conversion factor of 1.057 applied to correct for HCl salt.

### Container Closure System

Table 7 lists the packaging materials used in all studies.

TABLE 7
Container Closure System

| Material | Function | Description |
|---|---|---|
| Film: 165 mm 5 layer AAB | Base film | Aluminum-polymer laminate |
| Sachet: 110 mm × 170 mm | Sachet | Aluminum, tie layer, polymer |
| Foil: 273: | Sealing Foil | Aluminum-polymer-paper |
| PR53 Spec P0066 | | laminate |

### Technical Risk Assessment

The technical risk assessment has been updated with the knowledge gained during these studies. Table 8 summarizes the findings.

TABLE 8
Technical Risk Assessment

| Technical Assessment | Risk Analysis | Update |
|---|---|---|
| High dose strengths and highly soluble | Risk to Zydis structure/collapse | The use of glycine as a structural enhancer was |
| API. | during freeze drying. | evaluated. It did not provide any structural benefit in |
| | | comparison with the formulations without glycine; |
| | | therefore it was not required for future studies. |
| | | The concentration of matrix formers gelatin and |
| | | mannitol were assessed during study 4 to determine a |
| | | structurally sound formulation suitable to be brought |
| | | forward into a technical batch. |
| | | 50 mg in 600 mg fill weight was selected which should |
| | | reduce risk. |
| Requirement for permeation enhancer | Limited experience of | Dodecyl Maltoside was successfully incorporated into |
| | permeation enhancers and | selected formulation. |
| | impact on Zydis formulation | |
| | structure and potential for | |
| | improvement in | |
| | bioavailability. | |
| Chemical stability | In-process and finished product. | Stability study ongoing. Initial assay and related |
| | | substances signify mixes are stable over 24 hour holding |
| | | period. |
| Taste | Taste properties unknown. API | Remains unknown until first in vivo study. |
| | will be immediately available to | |
| | taste in mouth. | |

### Residual Solvents

The residual solvent assessment for Zydis ® BHV-3500 at the feasibility stage of development is shown in Table 9.

TABLE 9
Residual Solvent Assessment

| | | | | | | Meet USP |
|---|---|---|---|---|---|---|
| | | Concentration | | Concentration | | requirements |
| Materials | Residual solvents | present | | limit | | (option 1) |
| Gelatin EP/USP/JP | Acetic Acid | ≤5000 | ppm | 5000 | ppm | Yes |
| (Fish HMW) | Isopropyl Alcohol | ≤5000 | ppm | 5000 | ppm | Yes |
| Mannitol EP/USP/JP | None present | N/A | | N/A | | Yes |
| BHV-3500* | Methyl t-butyl ether (MTBE) | ≤5000 | ppm | 5000 | ppm | Yes |
| | Acetone | ≤5000 | ppm | 5000 | ppm | Yes |
| | Dichloromethane (DCM) | ≤600 | ppm | 600 | ppm | Yes |
| | Ethanol | ≤5000 | ppm | 5000 | ppm | Yes |
| | n-Heptane | ≤5000 | ppm | 5000 | ppm | Yes |
| | Dimethylformamide (DMF) | ≤880 | ppm | 880 | ppm | Yes |
| | Dimethylacetamide (DMA) | ≤1090 | ppm | 1090 | ppm | Yes |
| Dodecyl | Dichloromethane (DCM) | ≤600 | ppm | 600 | ppm | Yes |
| Maltopyranoside* | Methanol | ≤3000 | ppm | 3000 | ppm | Yes |
| | Isopropyl Alcohol | ≤5000 | ppm | ≤5000 | ppm | Yes |

*A new grade of DDM (Dodecyl Maltopyranoside DDMP) was required for GMP manufacture, this is the material included in the residual solvent assessment.

### Summary

Based on finished product testing results from study 5 and considering customer feedback, it is recommended to manufacture batch 24840/120/1 containing DDM at 0.25% w/w at a larger scale (approximately 5kg) prior to a bench scale clinical manufacture, to ensure manufacturability of the formulation.

Whilst this formulation maybe suitable for technical batch/clinical GMP manufacture further optimization/product characterization is strongly recommended after the first in human trial.

### Example 3

The purpose of this study was to develop a softgel formulation suitable to improve bioavailability of BHV-3500 for clinical trials. BHV-3500 is a molecule soluble in water but with a low permeability linked to its high polarity. The oral bioavailability of this compound is therefore fairly low. After execution of an OptiForm Solution Suite (OFSS) Bio program, a lead formulation was selected. This formulation is an unoptimized formulation as only a two-week stability study was performed. Moreover, as the lead formulation is a suspension, a thickener was needed to ensure content uniformity during encapsulation.

After the selection of a thickener, a compatibility study was performed to assess the stability of BHV-3500 in the different excipients. An informal stability study was then performed to confirm the excipient selection and adjust the formulation. This formulation was also tested in an animal PK study to evaluate the PK parameters of the formulation. Based on the PK and preliminary stability, one formulation was selected, four different batches of capsules with different drug loads were manufactured and placed on accelerated stability.

### Optiform Solution Suite Results

This program is the continuation of on OFFS Bio project, where the assessment of the increase of permeability using sodium caprate was made. This assessment was positive and allowed us to continue with the in-vivo testing of four different enabled formulations which would be able to release sodium caprate during their digestion in the intestine. Based on this in vivo evaluation, one formulation was selected as the formulation was able to improve the plasma concentration of BHV-3500 on oral administration and it did not show any significant degradation of the drug substance during a short term stability study (2weeks/40° C.).

The selected formulation composition is detailed in Table 10 below. This formulation contains a high level of medium chain mono-, di- and triglycerides that can release sodium caprate during digestion by the intestinal enzymes that could potentially increase the intestinal permeability of BHV-3500. The polysorbate helps to emulsify the formulation and optimize the kinetics of digestion. The API is suspended in the formulation.

TABLE 10
Placebo Formulation Composition after OFSS Bio (BHV-002)

| Chemical Name | Ratio(%) | Function |
|---|---|---|
| Medium Chain Triglycerides | 65 | Primary source of permeation |
| | | enhancer |
| Medium Chain Mono- and | 25 | Secondary source of |
| Diglycerides | | permeation enhancer and |
| | | emulsifier |
| Polysorbate 80 | 10 | Emulsifier |

In order to maintain the drug substance in suspension, there was a need to increase the viscosity using a thickener.

No compatibility study and no long-term stability study were conducted on this formulation, as the drug load was not established.

### Formulation Optimization

### Thickener Selection

Different thickeners were tried at different amounts to increase the viscosity of the mix presented in Table 10. As the purpose of this study was just to identify a good candidate and an approximate amount, a visual observation of the suspension was made for the comparison of the different excipients involved. Table 11 summarizes the results. Because of its better ability to increase the viscosity, Aerosil 200 was selected at approximately 5% level.

TABLE 11
Visual Assessment at Room Temperature of the
Thickeners Evaluated and their Ranking

| Component | Viscosity | Observation |
|---|---|---|
| Plasdone K90 (5%) | Similar (0) to initial | White precipitate at |
| | formulation | bottom of vial |
| Lecithin (5%) | Similar (0) to initial | Darker color |
| | formulation | |
| Geloil (15%) | Increase (+) | Turbidity increases, |
| | | a blur mix |
| Aerosil 912R (5%) | Increase (+) | Clear solution with |
| | | similar color |
| Aerosil 200 (5%) | Increase (++) | Turbidity increases, |
| | | a blur mix |
| Aerosil 200 (10%) | Viscosity max (++++) | Sample lost fluidity |

### Compatibility Study

A compatibility study was performed to identify any major API degradation in presence of the excipients. A list of excipients was identified based on the formulation selection, as well as common shell components. Additional samples were prepared with hydrophilic excipients to incorporate a 5% water spike to evaluate the effect of possible water migration from the shell to the fill, which typically is dependent on the hydrophilicity of the excipients. The water was added to the excipient prior the addition of the APL Two (2) to five (5) mg of API was mixed with approximately Ig of each excipient or a mixture of water+excipient. All samples were prepared in glass vial closed with a plastic screw cap, mixed via vortex for at least 5 minutes and allowed to sit in an oven set at 40° C. for 2 timepoints (2, 3, 4 or 5 weeks as shown below). The samples were analyzed for assay and related substances by HPLC. Two main impurities were identified and included in this evaluation (RRT=0.43 and RRT=1.06). The results of this study are detailed in Table 12. After this first study was performed, alternate excipients were tested in order to identify excipients with better compatibility. The results of this study are detailed in Table 13.

TABLE 12
Compatibility Study of BHV-3500 Stored at 40° C. in Glass Vials

| | 2 weeks | | | 3 weeks | | | 5 weeks | | |
|---|---|---|---|---|---|---|---|---|---|
| | | Main | 2^{nd} | | Main | 2^{nd} | | Main | 2^{nd} |
| | % | impurity | impurity | % | impurity | impurity | % | impurity | impurity |
| | Label | RRT = | RRT = | Label | RRT = | RRT = | Label | RRT = | RRT = |
| Sample | claim | 1.06** | 0.43** | claim | 1.06** | 0.43** | claim | 1.06** | 0.43** |
| Tween 80 | 97.0 | 0.68 | 0.04 | | | | 70.3 | 9.60 | 0.03 |
| Tween 80 + water | 89.7* | 1.14 | 0.05 | | | | 93.0 | 4.59 | 0.24 |
| Tween 80 + Gelatin | 91.0* | 0.8 | 0.04 | | | | 80.7 | 5.68 | 0.14 |
| strip | | | | | | | | | |
| Crodamol GMCC- | 81.2* | | 0.11 | | | | 79.4* | | 0.29 |
| SS | | | | | | | | | |
| Crodamol GMCC- | 66.56* | | 0.18 | 83.7* | | 0.15 | | | |
| SS + Gelatin strip | | | | | | | | | |
| Miglyol 812N | 101.8 | | | | | | 97.0 | | |
| Miglyol 812N + | 96.4 | | | 104.3 | | 0.03 | | | |
| Gelatin strip | | | | | | | | | |
| Sesame oil | 101.0 | | | | | | 96.6 | | |
| Sesame oil + Gelatin | 97.7 | | | 99.7 | | | | | |
| strip | | | | | | | | | |
| Formulation BHV- | 102.3 | 0.52 | 0.04 | | | | 99.6 | 0.81 | 0.05 |
| 002 | | | | | | | | | |
| Formulation BHV- | 102.7 | 0.25 | 0.07 | | | | 98.6 | 0.38 | 0.19 |
| 002 + water | | | | | | | | | |
| Formulation BHV- | 101.9 | 0.35 | 0.02 | 98.5 | 0.84 | 0.15 | | | |
| 002 + Gel | | | | | | | | | |
| Formulation BHV- | 102.4 | 0.24 | 0.02 | | | | 99.3 | 0.5 | 0.12 |
| 002 + DDM | | | | | | | | | |
| Formulation BHV- | 100.4 | 0.29 | 0.07 | | | | 97.7 | 0.5 | 0.20 |
| 002 + DDM + water | | | | | | | | | |
| Formulation BHV- | 99.9 | 0.30 | 0.04 | 97.5 | 0.24 | 0.15 | | | |
| 002 + DDM + Gelatin | | | | | | | | | |
| strip | | | | | | | | | |
| Formulation BHV- | 90.4 | 0.27 | 0.05 | | | | 93.8 | 0.43 | 0.19 |
| 002 + DDM + serosil | | | | | | | | | |
| Formulation BHV- | 82.9* | | 0.05 | | | | 90.2* | 0.17 | 0.21 |
| 002 + DDM + | | | | | | | | | |
| serosil + water | | | | | | | | | |
| Formulation BHV- | 84.1* | 0.36 | 0.07 | 87.0* | 0.13 | 0.10 | | | |
| 002 + DDM + | | | | | | | | | |
| serosil + gelatin strip | | | | | | | | | |

*Low assay value can be explained by sample preparation issues
**% of area of main peak

As some excipients seems to induce some degradation of the API, alternative excipients were evaluated. Tween 80 was replaced by another grade (Tween 80 HP) and was labeled as in our system using the generic name Polysorbate 80. This grade is a low moisture, low peroxide version of Tween 80. Labrasol ALF was investigated as a possible replacement for Crodamol GMCC-SS. Lecithin, Symperonic PE (ethylene oxide/propylene oxide block copolymer), and Kolliphor EL were also tested as an alternative to Tween 80. A sample with a solution of gelatin at 5% in water was added to assess further the compatibility with the shell of the capsule. As the degradation occurs mainly in excipients prone to induce oxidation some samples were also tested with a supplement of vitamin Eat a ratio of 1% w/w in the excipient.

TABLE 13
Compatibility Study at 40° C. with Additional Excipients in Glass Vials

| | 2 weeks | | | 4 weeks | | |
|---|---|---|---|---|---|---|
| | % | Main | 2nd | | Main | 2nd |
| | Label | impurity | impurity | Label | impurity | impurity |
| Sample | claim | RRT = 1.06 | RRT = 0.43 | claim | RRT = 1.06 | RRT = 0.43 |
| Polysorbate 80 | 100.8 | 0.51 | 0.03 | 97.7 | 2.62 | 0.07 |
| Polysorbate 80 + Vit E | 93.4 | 0.22 | 0.02 | 104.8 | 0.44 | |
| Labrasol ALF | 93.3 | 4.47 | 0.02 | 0.0 | API fully degraded | |
| Labrasol ALF + Vit E | 96.6 | | 0.02 | 100.7 | | 0.04 |
| Phosal (Lecithin) | 94.0 | 1.53 | 0.26 | 84.0 | 7.35 | 0.57 |
| Symperonic PE | 99.3 | 0.75 | | 91.6 | 2.05 | 0.03 |
| Kolliphor EL | 95.2 | 2.25 | | 89.3 | 4.19 | |
| Gelatin 5% | 99.7 | 0.05 | 0.06 | 98.9 | 0.06 | 0.08 |

The new grade of Polysorbate 80 was able to limit the degradation, and with the presence of vitamin E further improvement of the stability of the API was observed. The Labrasol was not useful to replace the Crodamol GMCC-SS as it induces a full degradation of the API in 4 weeks, but here again the addition of 1% of vitamin E prevented the degradation of the APL. The other excipients were also not able to decrease the degradation rate of the APL. Therefore, it was decided to move forward with formulation BHV-002, with Aerosil 200 as a thickener, and use of Polysorbate 80. This formulation was later called BHV-007, see Table 14 for the details.

Propylene glycol dicaprylate (Labrafac-PG) is currently listed on FDA's inactive ingredients list (IIG) for topical use (10% w/w, CAS 7384987, UNII 581437HWX2). Labrafac-PG is not currently listed in the FDA's IIG database for oral delivery but is similar to the IIG listed glyceryl monostearate Crodamol GMCC-SS where instead of Medium Chain Mono- and Diglycerides, it is Propylene glycol dicaprylate/dicaprate (in which the backbone of glycerol of the Crodamol GMCC-SS is substituted by a propylene glycol for Labrafac-PG). The drug load of 50 rng/g was decided based on the PK study performed by Biohaven.

TABLE 14
Theoretical Compositions of Different
Formulations for Stability Evaluation

| | Formulations | | | | | |
|---|---|---|---|---|---|---|
| Excipients | BHV-007 | | BHV-008 | | BHV-009 | BHV-010 |
| Miglyol 812N | 61.75% | | 61.1% | | 61.75% | 61.1% |
| Crodamol | 23.75% | | 23.5% | | | |
| GMCC-SS | | | | | | |
| Polysorbate 80 | 9.5% | | 9.4% | | 9.5% | 9.4% |
| Aerosil 200 | 5.0% | | 5.0% | | 5.0% | 5.0% |
| Vitamin E | — | | 1% | | — | 1% |
| Labrafac | | | | | 23.75% | 23.5% |
| Drug Load | 50 | mg/g | 50 | mg/g | 50 mg/g | 50 mg/g |
| Mielyol 812N | 555.75 | mg | 549.9 | mg | 555.75 | 549.9 |
| Crodamol | 213.75 | mg | 211.5 | mg | 0 | 0 |
| GMCC-SS | | | | | | |
| Polysorbate 80 | 85.5 | mg | 84.6 | mg | 85.5 | 84.6 |
| Aerosil 200 | 45 | mg | 45 | mg | 45 | 45 |
| Vitamin E | | | 9 | mg | | 9 |
| Labrafac | | | | | 213.75 | 211.5 |
| Drug Load | 50 | mg/g | 50 | mg/g | 50 mg/g | 50 mg/g |

### Stability Study

To confirm the formulation, a stability study at 40° C. was performed with formulations BHV-007, BHV-008, BHV-009 and BHV-010. The results are detailed in Table 15.

TABLE 15
Results of the Stability Study at 40° C. in Glass Vials

Impurities

| | 4 weeks | 8 weeks | 12 weeks |
|---|---|---|---|
| BHV-007 | No impurity >0.05% | RRT 1.093: 0.17% | RRT 1.108: 0.29% |
| | of main peak | of main peak | of main peak |
| BHV-008 | No impurity >0.05% | No impurity >0.05% | RRT 0.562: 0.05% |
| | of main peak | of main peak | of main peak |
| BHV-009 | No impurity >0.05% | RRT 1.090: 0.37% | RRT 0.973: 0.08% |
| | of main peak | of main peak | of main peak |
| | | | RRT 1,118: 0.39% |
| | | | of main peak |
| BHV-010 | No impurity >0.05% | No impurity >0.05% | No impurity >0.05% |
| | of main peak | of main peak | of main peak |

Formulation BHV-008 was selected to continue the project since it only has 0.05% of impurity after 12 weeks at 40° C. This formulation was used in a PK study in dogs with two drug loads: 25 mg and 50 mg of BHV-3500 in 900 mg of vehicle respectively. The formulation was filled into hard gelatin capsules and these capsules were dipped into the enteric coating dispersion. 50 mg load provided the expected PK profile in animal model but it should be noted that both 25 and 50 mg were dose proportional in that animal study.

### Final Formulation Lead Selection and Prototype Manufacture

### Animal PK Study Results from Lead Formulations

The human PK profile of BHV-3500 that is known to be clinically active in the treatment of migraine was identified in a large Phase 2/3 dose-ranging clinical trial testing 5, 10 and 20 mg intranasal unit doses, and where 10 mg was identified as the lowest fully effective dose. A number of oral PK studies were conducted in dogs to evaluate how different excipients influenced the oral PK of BHV-3500. The formulation selection of BHV-008 was based on allometric scaling of dog oral PK which showed that projected human exposures for oral delivery were at or above the clinical exposures from 10 mg intranasal BHV-3500.

### Formulations Selected

The last PK study determined that the optimum drug load was 50 mg per 900 mg of formulation. Nevertheless, it implied quite large capsules to be manufactured. These capsules as they are enteric coated, will not rupture before they reach the upper intestine and therefore because of their size could be retained for a long time in the stomach. To limit this effect, it was decided to manufacture different sizes of capsules. The quantity of Aerosil 200 was slightly decreased to take in account the contribution of the API in the final viscosity of the mix. Previous assessments of this quantity were made on placebo as it required a large amount of formulation to be prepared. An additional study was made with a progressive decrease of Aerosil 200 and show that the use of around 4.4% of Aerosil still maintains a good viscosity of the fill formulation.

Two different strategies were followed, one consisted of dividing the dose into two capsules, the other one was to decrease the quantity of formulation to deliver the 50 mg of APL Table 16 details these different formulations.

TABLE 16
Formulation Compositions of the Different Soft Gelatin Capsules Manufactured

| | Formulation A | Formulation A | Formulation B | Formulation C | Placebo |
|---|---|---|---|---|---|
| Component | 13 Oblong (mg) | 9.5 Oblong (mg) | (mg) | (mg) | 25 mg (mg) |
| Miglyol 812N | 552.6 | 276.3 | 414,45 | 276.3 | 291.6 |
| Crodamol GMCC- | 212.4 | 106.2 | 159.3 | 106.2 | 112.1 |
| SS | | | | | |
| Polysorbate 80 | 85.5 | 42.75 | 64.125 | 42.75 | 45.1 |
| Vitamin E (D.L. | 9.5 | 4.75 | 7.125 | 4.75 | 5.0 |
| alpha tocopherol) | | | | | |
| Aerosil 200 | 40.0 | 20.0 | 30.0 | 20 | 21.2 |
| BHV-3500 | 50.0 | 25.0 | 50.0 | 50.0 | 0 |
| Total | 950.0 | 475.0 | 725.0 | 500.0 | 475.0 |
| Size and shape of | 18 oblong | 9.5 oblong | 14 oblong | 9.5 oblong | 9.5 oblong |
| the capsules | | | | | |

### Mixing

The same mixing process was used for all the formulations. As a high level summary, utilizing a Becomix 2.5 L, the excipients were added starting with Polysorbate 80, the Crodamol GMCC-SS that was melted at 40° C. before weighing, and a portion of the Miglyol 812N. After a first mix, the Vitamin E was added and then the Aerosil 200 was added in 3 different portions to ensure homogeneity (if the Aerosil is added in one single step, it would be difficult to disperse it). The API was weighed in the isolator and the second half of Miglyol was added to prepare a slurry. This slurry was added to the contents of the Becomix to finalize the formulations.

For the placebo, the excipients, Polysorbate 80, Crodamol GMCC-SS, and Miglyol were successively added and mixed. After mixing for few minutes, the Vitamin E was added, then the Aerosil 200, here again in three different portions.

### Encapsulation

The fill materials were encapsulated using the lab scale encapsulation machine. White opaque size 18 oblong (Die: G18BE, Single Pocket), or size 14 oblong (Die: G14BF, Single Pocket) or size 9.5 oblong (Die: G9.5BC, Single Pocket) capsules were produced with minimal issue. In-process fill weights were taken approximately every 3 to 10 minutes throughout encapsulation. Capsules were weighed and then emptied (using ether and methanol to clean) prior to weighing the empty shells. The resulting fill weights are shown in Table 17. Each weight represents the weight of one capsule taken at different intervals during encapsulation (at least every five minutes). As the batch size are different, the number of measurements were variable.

TABLE 17
In-process Fill Weight Measurements

| | Fill Weight | Fill Weight | Fill Weight | Fill Weight |
|---|---|---|---|---|
| Product | Check 1 | Check 2 | Check 3 | Check 4 |
| Formulation A | Fill weight Target: 0.950 g | | | |
| 18 oblong | Range: 0.920-0.980 g | | | |
| 50 mg | | | | |
| (OET-10291023, | 0.949 g | 0.948 g | NT | NT |
| Lot 20MC-04) | | | | |
| Formulation A | Fill weight Target: 0.475 g | | | |
| 9.5 oblong | Range: 0.461-0.489 g | | | |
| 25 mg | | | | |
| (OET-10291024, | 0.489 g | 0.474 g | 0.480 g | 0.473 g |
| Lot 20MC-05) | | | | |
| Formulation B | Fill weight Target: 0.725 g | | | |
| (OET-10291025, | Range: 0.703-0.747 g | | | |
| Lot 20MC-06) | 0.713 g | 0.742 g | 0.727 g | NT |
| Formulation C | Fill weight Target: 0.500 g | | | |
| (OET-10291026, | Range: 0.485-0.515 g | | | |
| Lot 20MC-07) | 0.501 g | 0.501 g | 0.509 g | NT |
| 25 mg Placebo | Fill weight Target: 0.475 g | | | |
| (OET-10291081, | Range: 0.461-0.489 g | | | |
| Lot 20MC-39) | 0.477 g | 0.484 g | 0.491 g* | 0.464 g |

*Action taken to reduce fill weight
NT: Not taken

### Drying

During the drying phase the capsules were tested for their hardness (five capsules per timepoint) and were found to be within specification (8-10N) after 4 to 6 days (Table 18). The fill moisture was also assessed in the placebo, the values are an average of two measurement, each measurement is made after mixing of the fill of at least 3 capsules (Table 19).

TABLE 18
Prototype Capsules Average Hardness

| | Day 1 | Day 2 | Day 3 | Day 4 | Day 5 | Day 6 |
|---|---|---|---|---|---|---|
| [symbol] | Hardness | Hardness | Hardness | Hardness | Hardness | Hardness |
| 20MC-04 | | | 5.7 | | 8.1 | |
| 20MC-05 | | | 6.9 | | 9.8 | |
| 20MC-06 | 4.2 | | 6.8 | 7.3 | 8.2 | |
| 20MC-07 | | 7.6 | | 8.6 | | |
| 20MC-39 | | 6.5 | 7.5 | 7.4 | | 8.5 |

[symbol]

indicates data missing or illegible when filed

TABLE 19
Prototype Placebo Capsules Average Fill Moisture Results

| | Pre | Post | | | | | |
|---|---|---|---|---|---|---|---|
| Batch | encapsulation | encapsulation | Day 1 | Day 2 | Day 3 | Day 4 | Day 6 |
| 20MC-39 | 0.321% | 0.903% | 1.593% | 1.102% | 1.188% | 0.831% | 0.965% |

### Coating

The different batches were coated in a pan coater with a pH sensitive coating to enable a protection of the capsules in the stomach and a release in the intestine. The suspension formula of the coating is described in the Table 20. The suspension was prepared at room temperature by dispersing the polymer (AQUAPOLISH P CLEAR 792.03E) in water and then adding the plasticizer (propylene glycol) in a second step.

TABLE 20
Coating Suspension Formula

| | | Theoretical Planning |
|---|---|---|
| | Quantity Based | Weight of |
| | On Dry Polymer | Suspension Per Batch |
| Item Description | (%) | [symbol] |
| AQUAPOLISH P | — | 250.00 |
| CLEAR 792.03E | | |
| Propylene Glycol, | 20.00 | 50.00 |
| USP, EP | | |
| Purified Water, | — | 1,700.00 |
| USP, EP, JP | | |
| Or Deionized Water | | |

[symbol]

indicates data missing or illegible when filed

Stirring of the coating suspension was maintained during the whole process. The coating was sprayed onto the capsules in a pan coater with a targeted weight gain of at least 6% (see Table 21 for the actual value of weight gain).

TABLE 21
Actual Weight Gain on Coating of Capsules

| | Item Description of | |
|---|---|---|
| Item Description of | corresponding coated | Weight gain of |
| uncoated capsules | capsules | coated capsules |
| Formulation A 18 oblong | Formulation A 18 oblong Coated | 6.9% |
| (OET-10291023, Lot 20MC-04) | (OET-10291061, Lot 20SC-05) | |
| Formulation A 9.5 oblong | Formulation A 9.5 oblong Coated | 6.3% |
| (OET-10291024, Lot 20MC-05) | (OET-10291062, Lot 20SC-06) | |
| Formulation B | Formulation B Coated | 6.2% |
| (OET-10291025, Lot 20MC-06) | (OET-10291063, Lot 20SC-07) | |
| Formulation C | Formulation C Coated | 6.3% |
| (OET-10291026, Lot 20MC-07) | (OFT-10291064, Lot 20SC-08) | |
| 25 mg Placebo | 25 mg Placebo Coated | 6.6% |
| (OET-10291081, Lot 20MC-39) | (OET-10291095, Lot 20SC-09) | |

### Prototype Stability

The informal stability results of the prototype batches are outside the scope of this development report and will be reported separately. As part of development process, the informal stability results may be used to further define the specification limits appropriate for this product. The draft T=0 results were available at the time of this report and shown in Table 22. These data is comparable to what was seen in the formulation fill stability study performed earlier. T=1 month results for (OET-10291062, Lot 20SC-06) are shown in Table 23, T=0 results are also copied in this table to allow comparison of these two timepoints. All the impurities reported are within the specifications.

TABLE 22
Initial Data for the Active Prototype Capsules.

| Test | | (OET-10291061, | | (OET-10291062, | | (OET-10291063, | | (OET-10291064, | |
|---|---|---|---|---|---|---|---|---|---|
| (Specification) | | Lot 20SC-05) | | Lot 20SC-06) | | Lot 20SC-07) | | Lot 20SC-08) | |
| Disintegration | Condition | 0.1 NHC | pH = 6.8 | 0.1 NHC | pH = 6.8 | 0.1 NHC | pH = 6.8 | 0.1 NHC | pH = 6.8 |
| | | | buffer | | buffer | | buffer | | buffer |
| | Average | — | 22 | — | 17 | — | 18 | — | 18 |
| | | | Minutes | | Minutes | | Minutes | | Minutes |
| | Maximum | — | 24 | — | 19 | — | 19 | — | 20 |
| | | | Minutes | | Minutes | | Minutes | | Minutes |

| Assay | | 100.6% | 99.2% | 97.7% | 98.5% |
|---|---|---|---|---|---|
| Content Uniformity | | Average = 96.8% | Average = 97.2% | Average = 99.1% | Average = 95.2% |
| | | % RSD = 0.9 | % RSD = 0.7 | % RSD = 1.9 | % RSD = 3.7 |
| | | AV = 3.7 | AV = 30 | AV = 4.5 | AV = 11.7 |
| Related | RRT | ND | 0.15 | ND | ND |
| Substances | 0.496 | | | | |
| | RRT | 0.14 | ND | 0.11 | ND |
| | 0.504 | | | | |
| | BMS | ND | ND | ND | ND |
| | 748668 | | | | |
| | BMS | 0.14 | 0.13 | 0.12 | 0.11 |
| | 776765 | | | | |
| | BMS | ND | ND | ND | ND |
| | 769006 | | | | |
| | RRI | 0.06 | 0.06 | 0.07 | 0.06 |
| | 1.048 | | | | |
| | BMS | ND | ND | ND | ND |
| | 769740 | | | | |
| | RRT | 0.16 | 0.15 | 0.33 | 0.10 |
| | 1.257 | | | | |
| | RRT | 0.07 | 0.07 | 0.08 | 0.06 |
| | 1.329 | | | | |
| | RRT | 0.09 | 0.08 | 0.12 | 0.08 |
| | 1.335 | | | | |
| | RRT | ND | 0.11 | ND | 0.11 |
| | 1.458 | | | | |
| | Total | 0.66% | 0.75% | 0.63% | 0.52% |

ND: Not detected

The capsules were stored in aluminum bags, then sampled in plastic bags for delivery to Analytical Research and Development. As they were not sealed, the capsules acclimated to the humidity of the laboratory, absorbing moisture from the atmosphere. For future batches, the hardness range for the capsule release should be further evaluated.

TABLE 23
Initial Data and T = I month
for OET-10291062, Lot 20SC-06 Capsules.

| | | (OET 10291062, | | (OET 10291062, | |
|---|---|---|---|---|---|
| Test | | Lot 20SC 06) | | Lot 20SC 06) | |
| (Specification) | | T = 0 | | T = 1 month | |
| Disinte- | Condition | 0.1 NHCl | pH = | 0.1 NHCl | pH = |
| gration | | | 6.8 buffer | | 6.8 buffer |
| | Average | — | 17 | — | 24 |
| | | | Minutes | | Minutes |
| | Maximum | — | 19 | — | 27 |
| | | | Minutes | | Minutes |

| Assay | | 99.2% | 98.9% |
|---|---|---|---|
| Content | | Average = 97.2% | — |
| Uniformity | | %RSD = 0.7 | — |
| | | AV = 3.0 | — |
| | RRT0.315 | ND | 0.06 |
| | RRT0.338 | ND | 0.10 |
| Related | RRT | 0.15 | 0.17 |
| Substances | 0.496 | | |
| | RRT | ND | ND |
| | 0.504 | | |
| | BMS | ND | ND |
| | 748668 | | |
| | RRT0.972 | | 0.07 |

| | | |
|---|---|---|
| BMS | 0.13 | 0.07 |
| 776765 | | |
| BMS | ND | LTLOQ |
| 769006 | | |
| RRT | 0.06 | ND |
| 1.048 | | |
| BMS | ND | ND |
| 769740 | | |
| RRT | 0.15 | 0.17 |
| 1.257 | | |
| RRT | 0.07 | ND |
| 1.329 | | |
| RRT | 0.08 | 0.08 |
| 1.335 | | |
| RRT | 0.11 | ND |
| 1.458 | | |
| Total | 0.75% | 0.72 |

### Conclusion

BHV-3500 has been successfully formulated and encapsulated in soft gelatin capsules. Available data suggests that the prototype formulations are adequately stable, which allows us to start to manufacture prototype capsules to be assessed for their stability in ICH conditions, for method development. The formulation A 9.5 Oblong (OET-10291062) was selected for subsequent animal oral safety studies to confirm bioavailability and evaluate repeat-dose safety of the API delivered with these capsules and to move forward with a GMP manufacture for a Phase O clinical trial. Informal stability studies are ongoing to further understand the stability of the prototype formulation.

### Example 4

The objective of this study is to manufacture cGMP Phase O non-commercial clinical batches of Biohaven's BHV-3500 Placebo 9.5 oblong and BHV-3500 25 mg 9.5 oblong softgels. One batch of fill material for each strength is manufactured at a theoretical batch size of 1900.0 g using a 2.5L Becomix vessel. Each fill mix is used to encapsulate one batch of the corresponding strength of BHV-3500 active and placebo softgels at theoretical batch sizes of 4000 units for each product (Table 24). The capsules are then coated using a pan coater.

TABLE 24
BHV-3500 Finished Product OET Numbers

| | | |
|---|---|---|
| Bulk Product Name | BHV-3500 25 mg | BHV-3500 Placebo |
| | 9.5 oblong | 9.5 oblong |
| Bulk Product | OET-10275670 | OET-10275671 |
| Number | | |

### Scope

The scope of this protocol is limited to the manufacture of one batch of each strength of BHV-3500 softgels (Placebo and 25 mg). The purpose of manufacturing this cGMP batches is to provide the clinical trial material (CTM) for a Phase O study in humans and to conduct formal stability studies.

### Background

This product is intended to treat migraine and will be used in a Phase O clinical trial to be performed in the United States by the customer. The manufacture of non GMP batches of the active and the placebo product was performed for use in informal stability study (see PD-20-049R for details).

### Responsibilities

Product Development Technical Lead is responsible for writing the protocol, training all personnel involved in the manufacture of the batch, and overseeing the execution of the batch ns needed.

Product Development Management, Operations Manager, Analytical R&D/Quality Control, Validation, Quality Assurance, and Biohaven (Customer) or designee is responsible for approving the protocol.

Production Operators (Material Preparation, Encapsulation, and Finishing operators) and/or Product Development Technicians/Specialists are responsible for the execution of the batches listed in the protocol.

Validations are responsible for issuing a cleaning verification protocol and to annotate the batch record. Quality Assurance is responsible for final approval of the protocol.

### Instructions

The softgel manufacturing process is divided into seven separate modules which will be explained in the following sections:

### 1) Fill Material Preparation

### 2) Gel Mass Preparation

### 3) Encapsulation

### 4) Softgel Drying

### 5) Softgel In-Process Finishing/Packaging

### 6) Softgel Coating

### 7) Softgel Bulk Packaging

The manufacturing instructions for each of these modules can be found in the BHV-3500 25 mg

9.5 oblong softgels, OET-10275670 and BI]V-3500 Placebo 9.5 oblong softgels OET-10275671 master batch records.

### Fill Material Preparation

BHV-3500 25 mg 9.5 oblong Softgels contain the active pharmaceutical ingredient (API), BHV-3500, suspended in Miglyol 8 l 2N, Crodamol GMCC-SS, Polysorbate 80, Colloidal Silicon Dioxide and D,L alpha tocopherol, at a concentration of 5.263% w/w BHV-3500 (corresponding to 25 mg) and are encapsulated at a theoretical fill weight of 475 mg per softgel. BHV-3500 Placebo 9.5 oblong softgels is the matching placebo of the active softgels. Refer to Table 25 and Table 26 for both Master Formula.

The target weight of BHV-3500 is adjusted for potency to 100% during weigh off based on Assay Purity (Supplier COA). Miglyol 812N is be compensated for the potency adjustment as per the issued batch record.

TABLE 25
BHV-3500 25 mg 9.5 oblong Master Formula

OET-10275670 BHV-3500 25 mg 9.5 oblong
Theoretical Batch Size: 4,000 Coated Softgels

| | | | Active | Amount per | Amount per |
|---|---|---|---|---|---|
| Fill Material | Item | Function | w/w % | Softgel (mg) | Batch (g) |
| Medium Chain | OET- | Suspending | 58.168 | 276.3 ¹ | 1105.2 |
| Triglycerides | 00303407 | agent | | | |
| (Miglyol 812N), NF | | | | | |
| Glycerol Monocaprylo | OET- | Surfactant | 22.358 | 106.2 | 424.8 |
| Caprate (Cromadol | 003010092P1 | | | | |
| GMCC-SS), EP | | | | | |
| Polysorbate 80, NF, | OET- | Surfactant | 9.000 | 42.75 | 171.0 |
| EP | 00304226 | | | | |
| Colloidal Silicon | 003010080P1 | Thickener | 4.211 | 20.0 | 80.0 |
| Dioxide(Aerosil 200), | | | | | |
| NF | | | | | |
| D,L alpha tocopherol, | OET- | Antioxidant | 1.000 | 4.75 | 19.0 |
| USP | 00300590 | | | | |
| BHV-3500 | OET- | API | 5.263 | 25.0 ¹ | 100.0 |
| | 003085058P1 | | | | |
| Total | | | 100.0 | 475 mg | 1,900 g |

Coating Formulation Composition³

| | | | Coating | Applied Solids | |
|---|---|---|---|---|---|
| | | | Dispersion | Amount per | % w/w |
| Coating Material | Item | Function | w/w % | Softgel (mg) | Solids |
| AQUAPOLISH P CLEAR | OET- | Coating | 12.5 | 49.90 | 83.33 |
| 792.02 E | 003080002P1 | polymer | | | |
| Propylene Glycol, | OET- | Plasticizer | 2.5 | 9.98 | 16.67 |
| USP, EP | 00304185 | | | | |
| Purified Water, | OET- | Diluent | 85.02 | N/A² | N/A² |
| USP, EP, JP | 00308201 | | | | |
| Total | | | 100.0 | 59.88 | 100.00 |

¹ Weights are adjusted to take in account the potency of the BHV-3500
²Removed from processing
³Assumes a theoretical total softgel capsule weight of 998 mg before coating and a 6% target weight gain

TABLE 26
BHV-3500 Placebo 9.5 oblong Master Formula
OET-10275671 BHIV-3500 Placebo 9.5 oblong
Theoretical Batch Size: 4,000 Softgels

| | | | | Amount | |
|---|---|---|---|---|---|
| | | | | per | |
| | | | Active | Softgel | Amount per |
| Fill Material | Item | Function | w/W % | (mg) | Batch (g) |
| Medium Chain | OET- | Suspending | 61.389 | 291.6 | 1166.4 |
| Triglycerides | 00303407 | agent | | | |
| (Miglyol 812N), NF | | | | | |
| Glycerol Monocaprylo | OET- | Surfactant | 23.600 | 112.1 | 448.4 |
| Caprate(Cromadol | 003010092P1 | | | | |
| GMCC-SS), EP | | | | | |
| Polysorbate 80, NF, | OET- | Surfactant | 9.495 | 45.1 | 180.4 |
| EP | 00304226 | | | | |
| Colloidal Silicon | 003010080P1 | Thickener | 4.463 | 21.2 | 84.8 |
| Dioxide (Aerosil 200), | | | | | |
| NF | | | | | |
| D,L alpha tocopherol, | OET- | Antioxidant | 1.053 | 5.0 | 20.0 |
| USP | 00300590 | | | | |
| Total | | | 100.0 | 475 mg | 1,900 g |

Coating Formulation Composition²

| | | | Coating | Applied Solids | |
|---|---|---|---|---|---|
| | | | Dispersion | Amount per | % w/w |
| Coating Material | Item | Function | w/w % | Softgel (mg) | Solids |
| AQUAPOLISH P CLEAR | OET- | Coating | 12.5 | 49.90 | 83.33 |
| 792.02 E | 003080002P1 | polymer | | | |
| Propylene Glycol, | OBT- | Plasticizer | 2.5 | 9.98 | 16.67 |
| USP, EP | 00304185 | | | | |
| Purified Water, | OET- | Diluent | 85.01 | N/A¹ | N/A¹ |
| USP, EP, JP | 00308201 | | | | |
| Total | | | 100.0 | 59.88 | 100.00 |

¹Removed from processing
²Assumes a theoretical total softgel capsule weight of 998 mg before coating and a 6% target weight gain

The active fill solution is prepared in the 2.5 L Becomix closed mixing tank MV27 following the mixing instructions in the material preparation order section.

BHV-3500 is dispensed in the isolator as per the issued batch record.

In preparation for batch manufacturing, Glycerol Monocaprylo Caprate (Crodamol GMCC-SS-(MV)) must be liquified at approximately 40° C. (104° F.) in a hot box for at least 24 hrs and homogenized prior to weigh off.

### Gel Mass Preparation

For each individual batch, gel operators manufacture one (1) 004007 gel mass which is then color converted to 005007@91 IP (opaque white) for encapsulation.

### Encapsulation

One (I) batch of BHV-3500 25 mg 9.5 oblong and one (I) batch of BHV-3500 Placebo 9.5 oblong softgels are encapsulated at a theoretical batch size of 4,000 softgels each using an opaque white gel mass formula 005007@9 1 1 P. All encapsulation tooling information is listed in the OET-10275670 and OET- 10275671 master batch records. In-process fill weights/shell weights and seal thickness checks are performed as per the instructions in the master batch records. After encapsulation, the product is referred to as BHV-3500 25 mg 9.5 oblong softgels (Cores) and BHV-3500 Placebo 9.5 oblong softgels (Cores) because the enteric coating is be applied until later in the process.

Since these batches arc relatively small (4000 softgels) compared to commercial lots, the rejection rate could be higher therefore the reject limit of 3% as per SOP is not applied. As this product moves through the development, appropriate reject limits are established based on process behavior. Every effort is made to minimize rejects.

### SoftGel Drying

The shallow tray stacks containing the BHV-3500 25 mg 9.5 oblong softgels (Cores) and BI-IV-3500 Placebo 9.5 oblong softgels (Cores) are placed into drying tunnels. The softgels are dried in the tunnels on standard shallow trays until the individual softgel hardness values are within the specification range of 8.0 11.0 N. At daily intervals (12±2 hour) from the time the last stack of each day's encapsulation is completed, the last stack of each day's production per batch is sampled and tested per SOP (“In Process Hardness Determination Using Bareiss Durometer”). The hardness for the last stack from each day's encapsulation for each batch determine the release for inspection into deep trays for all the stacks completed for that day's production. The hardness results are recorded in the batch record.

After the softgels from the shallow tray stacks for each batch have met the hardness dying requirement, the softgels are transferred from the shallow trays to deep trays.

### SoftGel In-Process Finishing and Packaging

Softgel In-Process Finishing consists of manual capsule washing as per the issued batch record.

Complete 100% inspection after the washing process. Cull out softgels with defects and record in Scrap/Sample Reject section of the Batch Record.

One (1) Chemistry Retain Sample of uncoated softgels (Cores) is taken during this phase of the manufacturing process as per the issued batch record. The packaged softgels are put in deep trays before the coating step.

### SoftGel Coating

The functional coating suspension manufacture and softgel coating are performed as per the issued batch record in the West Wing room B0702, B0707. Labels for all the required samples arc provided in the batch record.

### SoftGel Bulk Packaging

The bulk coated softgels are bulk packaged in the final configuration as per the issued batch record. The bulk packaging configuration for all bulk coated softgels with the exception or the samples listed in the issued batch record is 100 softgels in a single sealed aluminum bag package. The sealed aluminum bags are put in carton, each carton contain a maximum or five bags. Sixteen (16) of these aluminum bags are then provided to Product development to be shipped to customer CRO to perform an animal study. This samples can be shipped prior to release as they can be used as GMP or non GMP material.

One (1) carton is prepared by packaging separately fifty (50) coated softgels. These fifty softgels (50) are packaged in five (5) separate aluminum bags with ten (10) softgel each (total of 50 softgels).

### Bulk Finished Product Release Testing

Bulk finished product release testing are performed according to the bulk finished product specification for BHV-3500 25 mg 9.5 oblong and for BHV-3500 Placebo 9.5 oblong. Product Development or designee fills out an RFA (request for analysis) for all tests required and submit the appropriate samples. A separate microbial limit testing (MLT) method validation (MV) sample, which is listed in the issued batch record is also submitted to the Micro group. This sample is used to perform the micro method validation prior to performing MLT testing required by the finished product specification. Three (3) composite samples are collected for each batch as follows:
One (1) J\R&D Chemistry Sample
   50 Softgels in ONE STERILE BAG packaged into an ALUMINUM BAG
One (I) Manufacturer Retain Sample
   75 Softgels in ONE STERILE BAG packaged into an ALUMINUM BAG
One (1) Microbiological Limits Release Testing Sample
   160 Softgels in ONE STERILE BAG packaged into an ALUMINUM BAG

### Inspection and AQL Testing

A representative sample of coated softgels is obtained by a Finishing Operator or designee and inspected per SOP “Acceptable Quality Limits (J\QL) Inspection” during bulk packaging. AQL Level II inspection results are recorded on “Acceptable Quality Limits (J\QL) Inspection Form”. This is annotated in the issued batch record in the finishing section for bulk coated softgels. As the defect of coating are similar to the defect that could occur on the shell of an uncoated softgel, the same criteria are applied (AQL Inspection book is used for this inspection). The sample size to be pulled is determined per SOP based on batch size, see Table 27.

TABLE 27
Sampling Plan for Normal Level Il Inspection

Defects Allowed (Ac)¹

| Lot Size | Sample Size | Critical | Major | Minor |
|---|---|---|---|---|
| (capsules) | (capsules) | (0.065%) | (0.65%) | (2.5%) |
| 501-1,200 | 80 | 0 | 1 | 5 |
| 1,201-3,200 | 125 | 0 | 2 | 7 |
| 3,201-10,000 | 200 | 0 | 3 | 10 |

¹Contact immediately Product Development (PD) and Quality Assurance (QA) if the number of defects is higher than the limits (Defects Allowed) of the table.

### Raw Material Release Testing

The API procured by Biohaven is used to manufacture the batch. The lot is tested and released as per current release specifications. Should the API not be completely released at the time of expected manufacture, a temporary change control (TCC) is evaluated as a potential process to manufacture in parallel with API release.

Excipients and packaging are fully released per Manufacturer's standard procedures by the Quality Control laboratory per the raw material specification for each item. Should excipients not be completely released at the time of expected manufacture, a temporary change control (TCC) is evaluated as a potential process to manufacture in parallel with excipient release.

### Example 5

Zavegepant (BHV-3500), Study BHV3500-107, Cohort 1 50 mg SoftGel QD (fast/fed), Preliminary PK Summary

Summary of Data collection for Zavegepant (BHV-3500) PK Analysis/Summary

Study BHV3500-107
Cohort 1 (Zavegepant (BHV-3500) 50 mg (2×25 mg) SoftGel administered orally QD fasting Days 1-10 days, fed (moderate fat meal Days 11-14)
Intensive PK—Day 1 (fasted, single dose), Day 10 (fasted, multiple dose), and Day 14 (fed, multiple dose)
Sparse PK (pre-dose and C8 hour)Day 3, 6, 9, and 12
PK Comparison Data

Single-dose: Study BHV3500-103, PK data after 50 mg×1 (2×25 mg) SoftGel administered orally (Treatment B)

Study BHV3500-107, Cohort 1 (Zavegepant 50 mg QD fasting/fed), Summary of PK
Day 1 Zavegepant PK* after a 50 mg single SoftGel dose under fasting conditions were similar to those observed after the same dose/formulation in Study BHV3500-103
Study 107, Cohort 1, Day 1: Tₘₐₓ=2.25 hours, Cₘₐₓ=5.3 ng/mL (1.4 to 13.8), and AUC_{0-inf}=20.0 ng*h/mL (9.5 to 47.8)
Study 103, Treatment B: Tₘₐₓ=1.75 hours, Cₘₐₓ=7.6 ng/mL (1.9 to 26.8) and AUC_{0-inf}=21.5 ng*h/mL (9.2 to 60.1)
Day 10 (steady state, fasting) vs. Day 1 PK* exposure showed minimal accumulation with similar Tₘₐₓ
accumulation ratio (AR) Cₘₐₓ=1.13 and AUC=1.35
Tₘₐₓ=2.0 hours, Cₘₐₓ=5.7 ng/mL (2.3 to 46.1), and AUC₀₋₂₄=20.4 ng*h/mL (6.7 to 75.4)

Steady-state exposures from 50 mg QD remain below those from 10 mg IN (Cₘₐₓ 13.0 ng/mL, AUC 33.0 ng*h/mL)
Day 14 (with food) vs. Day 10 (fasted) PK showed a substantial negative food effect
decrease in rate (˜72% in Cₘₐₓ) and extent (˜65% in AUC) of zavegepant absorption
delayed Tₘₐₓ(2 vs. 4 hours in fasted vs. fed, respectively)
Tₘₐₓ=3.6 hours, Cₘₐₓ=1.6 ng/mL (1.0 to 3.0), and AUC₀₋₂₄=7.3 ng*h/mL (3.7 to 16.6)
High PK variability and Cₘₐₓ and AUC (% CV>50%) was observed on each of intensive PK Days 1, 10, 14
Concentrations at 8 hours post-dose on Day 1, 3, 6, and 10 suggest steady state of zavegepant is achieved by Day 3 after multiple dosing of zavegepant in the fasted state

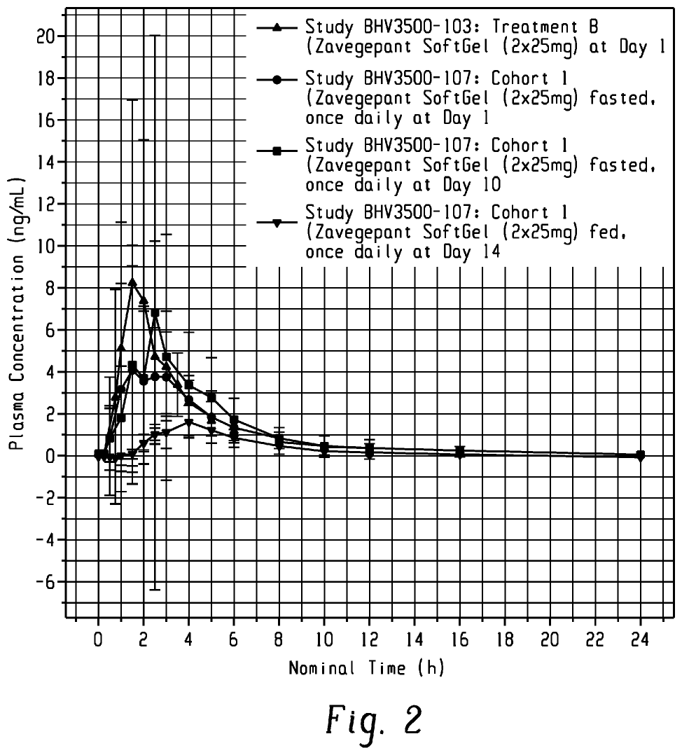

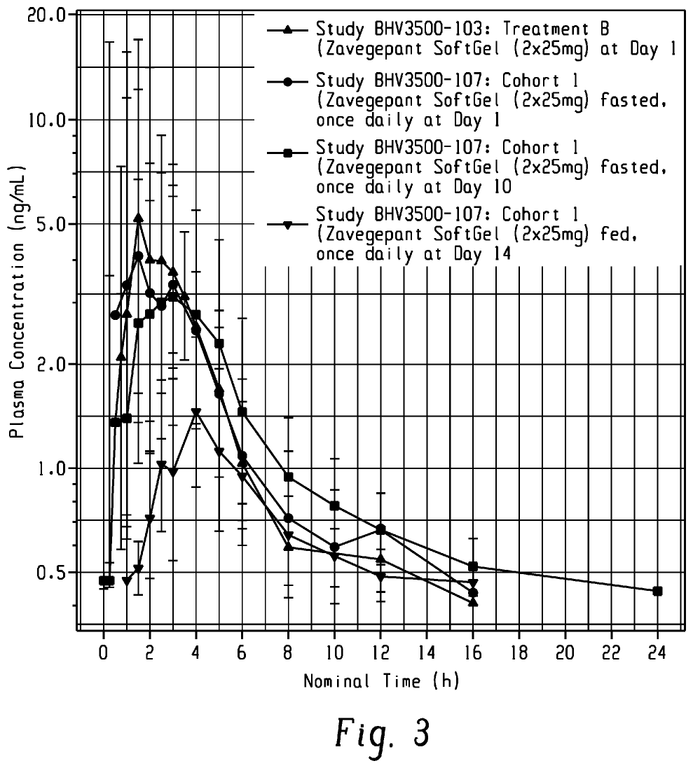

SoftGel 50 mg Mean (SD) PK profiles, Days 1, 10, 14 & Study 103 are shown in Table 28 and FIGS. 2 and 3.

TABLE 28
PK Summary 50 mg SoftGel × 1 (Study 107, Cohort 1, Day 1 and Study 103)

| | | Tmax | Cmax | AUCINF_obs | AUC0-24 | HL_Lambda_z |
|---|---|---|---|---|---|---|
| Study | | (h) | (ng/ml) | (h*ng/ml) | (h*ng/ml) | (h) |
| 103 | N | 12.0 | 12.0 | 12.0 | 12.0 | 12.0 |
| | Mean | 1.98 | 10.8 | 25.8 | 25.3 | 2.28 |
| | SD | 1.17 | 8.70 | 16.7 | 16.4 | 0.888 |
| | CV % | 59.1 | 80.8 | 64.7 | 65.0 | 38.9 |
| | Min | 0.500 | 1.86 | 9.21 | 8.65 | 1.48 |
| | Median | 1.75 | 7.06 | 19.1 | 18.8 | 1.98 |
| | Max | 4.00 | 26.8 | 60.1 | 59.0 | 4.74 |
| | Geometric Mean | 1.65 | 7.60 | 21.5 | 21.0 | 2.17 |
| 107 | N | 12.0 | 12.0 | 10.0 | 12.0 | 10.0 |
| | Mean | 2.42 | 6.32 | 22.6 | 19.1 | 2.66 |
| | SD | 1.18 | 3.68 | 12.3 | 12.4 | 1.15 |
| | CV % | 49.0 | 58.2 | 54.2 | 64.8 | 43.2 |
| | Min | 1.00 | 1.42 | 9.54 | 4.66 | 1.62 |
| | Median | 2.25 | 5.73 | 20.6 | 16.7 | 2.19 |
| | Max | 5.00 | 13.8 | 47.8 | 46.3 | 5.07 |
| | Geometric Mean | 2.18 | 5.29 | 20.0 | 15.8 | 2.48 |

Results of the multiple 50 mg Zavegepant QD SoftGel Dosing Fasting Day 10 are shown in Table 29.

TABLE 29

| | | | Cₘₐₓ | Tₘₐₓ | T [symbol] | AUC [symbol] | AUC [symbol] | Residual area | V [symbol] /F | C [symbol] /F |
|---|---|---|---|---|---|---|---|---|---|---|
| Cohort | Day | Subject | (ng/mL) | (h) | (h) | (h*ng/mL) | (h*ng/mL) | (%) | (L) | (L/ [symbol] ) |
| 1 | 1 | N | 12 | 12 | 1 [symbol] | 12 | 9.54 | 9 | 9 | 9 |
| | | Mean | 6.32 | 2.42 | [symbol] | [symbol] | 23. [symbol] | 9.7 [symbol] | 8 [symbol] 0 | 2650 |
| | | SD | 3.6 [symbol] | 1.18 | 1.15 | 12.1 | [symbol] 2.3 | 5.09 | 3350 | [symbol] 50 |
| | | Min | 1.42 | [symbol] | 1.6 [symbol] | 4.36 | 9.54 | [symbol] .62 | 3 [symbol] | [symbol] 0 |
| | | Median | 6.78 | 2.25 | 3.19 | 16.3 | 23.6 | 8.9 | 8450 | 23 [symbol] 0 |
| | | Max | 13.8 | [symbol] | 5. [symbol] 7 | 44. [symbol] | 47.8 | [symbol] 9.3 | 12500 | [symbol] |
| | | CV % | 58.14 | 48. [symbol] 9 | 43. [symbol] | [symbol] 5.86 | 5 [symbol] .85 | 52.01 | 38.53 | 50.92 |
| | | Geometric Mean | 5.29 | 2.18 | 3.48 | [symbol] 5.1 | 23.2 | [symbol] .57 | [symbol] 0 | 23 [symbol] |

| | | | Cₘₐₓ | Tₘₐₓ | T [symbol] | AUC [symbol] | AUC [symbol] | V [symbol] /F | C [symbol] /F |
|---|---|---|---|---|---|---|---|---|---|
| Cobort | Day | Subject | (ng/mL) | (h) | (h) | (h*ng/mL) | (h*ng/mL) | (L) | (L/ [symbol] ) |
| 1 | 10 | N | 11 | 11 | 10 | 11 | 11 | 10 | 11 |
| | | Mean | 9.71 | 2.69 | 6.8 [symbol] | 2 [symbol] .0 | 26. [symbol] | 19100 | [symbol] 8 [symbol] |
| | | SD | 13.1 | 1.24 | 4.64 | 20.6 | 2 [symbol] .1 | 7020 | 2260 |
| | | Min | 2.27 | 1.5 | 1.87 | 6.08 | 6.67 | 7480 | [symbol] |
| | | Median | 4.7 | 2 | 5.81 | 21.8 | 23.7 | [symbol] 93 [symbol] | 211 [symbol] |
| | | Max | 46.1 | 5 | 1 [symbol] | 72.8 | 75.4 | [symbol] 900 | 7 [symbol] |
| | | CV % | 135.04 | 47.91 | 68. [symbol] | 7 [symbol] .54 | 7 [symbol] .3 | 36.51 | 70.97 |
| | | Geometric Mean | 5.72 | 2.35 | 5.4 [symbol] | [symbol] .4 | 3 [symbol] .4 | [symbol] 7800 | [symbol] 45 [symbol] |

[symbol]

indicates data missing or illegible when filed

### Observed

High PK variability (>50%)
Minimal accumulation after multiple dosing:
Geometric mean accumulation ratio (AR) is 1.13 for Cₘₐₓ and 1.35 for AUC₀₋ₜ

Results of the multiple 50 mg Zavegepant QD SoftGel Dosing Fed Day 14 are shown in Table 30.

TABLE 30

| | | | Cₘₐₓ | Tₘₐₓ | T [symbol] | AUC [symbol] | AUC [symbol] | V [symbol] /F | C [symbol] /F |
|---|---|---|---|---|---|---|---|---|---|
| Cohort | Day | Subject | (ng/mL) | (h) | (h) | (h*ng/mL) | (h*ng/mL) | (L) | (L/ [symbol] ) |
| 1 | 10 | N | 11 | 11 | 10 | 11 | 11 | 10 | 11 |
| | | Mean | 9.7 [symbol] | 2.59 | 6.82 | 35.9 | 26.9 | 1 [symbol] 100 | 31 [symbol] |
| | | SD | 13. [symbol] | 1.24 | 4.64 | [symbol] .6 | [symbol] 1.1 | 7020 | [symbol] 2 [symbol] |
| | | Min | 2.27 | 1.5 | 1.87 | [symbol] 8 | [symbol] .67 | 74 [symbol] | 663 |
| | | Median | 4.7 | 2 | 5.81 | 21.8 | 2 [symbol] .7 | 3930 [symbol] | 213 [symbol] |
| | | Max | 46. [symbol] | 5 | 15.4 | 72.8 | 75.4 | 32900 | 749 [symbol] |
| | | CV % | [symbol] 35. [symbol] 4 | 47.91 | 68.09 | 7 [symbol] .54 | 78.3 | 36.81 | 70.97 |
| | | Geometric Mean | 5.72 | 2.35 | 5.43 | 19.4 | 20.4 | 17800 | 2450 |
| 1 | 14 | N | 9 | 9 | 7 | [symbol] | 9 | 7 | 9 |
| | | Mean | 1.7 [symbol] | 3.78 | 5. [symbol] 1 | 7.85 | 8.65 | 4 [symbol] 0 | 781 [symbol] |
| | | SD | [symbol] .779 | [symbol] .18 | 4.53 | 4.67 | 5. [symbol] | 2 [symbol] 0 | 3 [symbol] |
| | | Min | [symbol] .9 [symbol] | 2.5 | 3.3 | 3.32 | 3.66 | 143 [symbol] 0 | 3810 |
| | | Median | 1.48 | [symbol] | 3.37 | [symbol] .67 | 6.05 | [symbol] 2960 | 327 [symbol] |
| | | Max | 3.02 | [symbol] | [symbol] .5 | [symbol] 4.7 | 16.6 | 713 [symbol] 0 | 137 [symbol] 0 |
| | | CV % | [symbol] 4. [symbol] 6 | 31.32 | 86.5 | 59.54 | 59.5 | 50.31 | 50.95 |
| | | Geometric Mean | 1.62 | 3.63 | 4. [symbol] | 6.73 | 7.34 | 36 [symbol] 0 | 6810 |

[symbol]

indicates data missing or illegible when filed

Substantial decrease in rate and extent of absorption, delayed Tₘₐₓ, with moderate fat meal were observed.

Food Effect (Day 14/10): Ratio of Geometric Means and 90% CI are shown in Table 31.

TABLE 31
Ratios (Zavegepant 2 × 25 mg SoftGel, Cohort 1
Day 14/Zavegepant 2 × 25 mg SoftGel, Cohort 1
Day 10), 90% Geometric Confidence Intervals, Intra-
and Inter-Subject CV (%) and P-values for BHV3500-107 Study

| | [symbol] | | | [symbol] | | | | [symbol] |
|---|---|---|---|---|---|---|---|---|
| [symbol] | [symbol] | [symbol] | [symbol] | [symbol] | [symbol] | [symbol] | [symbol] | [symbol] |
| AUC [symbol] | [symbol] | [symbol] | [symbol] | 22. [symbol] | 50. [symbol] | [symbol] | [symbol] | 0.00 [symbol] |
| AUC [symbol] | [symbol] | [symbol] | [symbol] | 23.55 | 52.35 | [symbol] | [symbol] | 0.00 [symbol] |
| [symbol] | 5.72 | [symbol] | 28.31 | [symbol] 5.4 [symbol] | 52. [symbol] | [symbol] | NC | 0.00 [symbol] |

¹ Calculated using least-squares means according to the formula: exp (DIFFERENCE) * 100.
² 90% Geometric Confidence Interval (CI) calculated according to the formula: exp(DIFFERENCE ± t(dfResidual)* SEDIFFERENCE) * 100.
³ Calculated according to formula: SQRT (exp (MSE) − 1) * 100.
⁴ Calculated according to formula: SQRT (exp ((MSSUBJECT − MSE)/2) − 1) * 100.
LSM: Least Square Mean.
NC: Not Calculable [symbol]
indicates data missing or illegible when filed

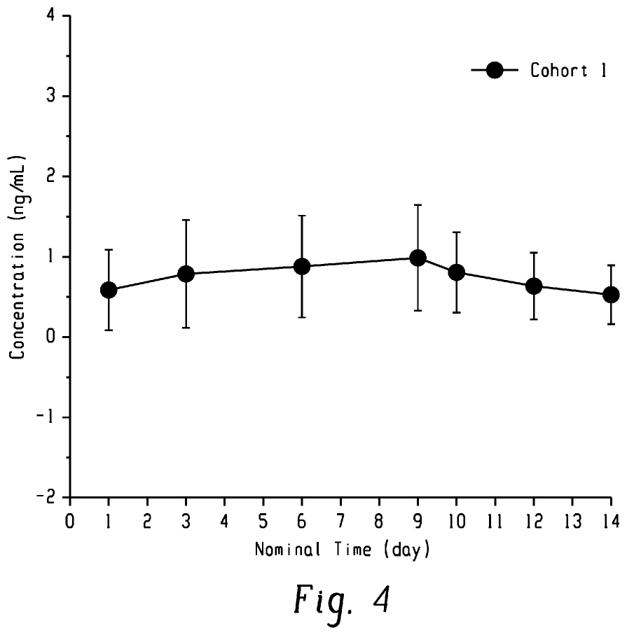

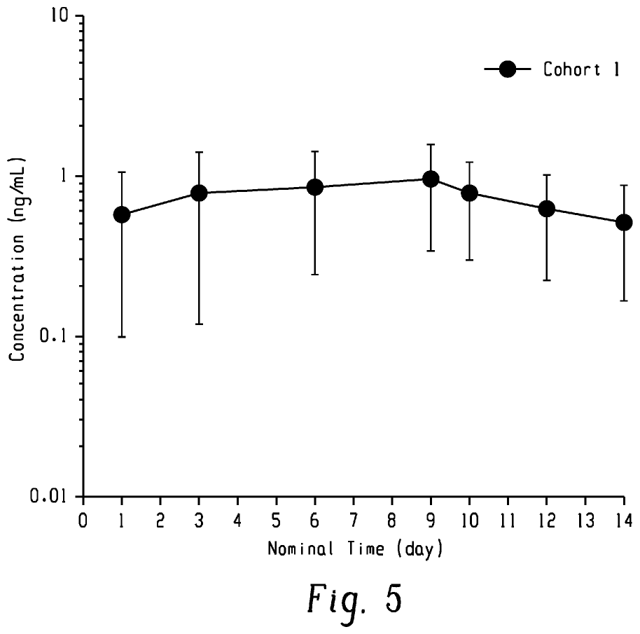

Cohort 1 50 mg QD Zavegepant Mean (±SD) C_{8 hour} Days 1, 3, 6, 9, 10, 12, and 14 are shown in FIGS. 4 and 5. 50 mg QD Fasting Days 1-10, Moderate Fat Meal Days 11-14.

Cohort 1 Zavegepant 50 mg QD Pre-dose and C_{8 hour} Days 1-14 are shown in Table 32.

TABLE 32

| | | Nominal Time (h) | | | | | | | | | | | | | |
|---|---|---|---|---|---|---|---|---|---|---|---|---|---|---|---|
| | | [symbol] (Pre-dose) | | | | | | | [symbol] | | | | | | |
| | | Day | | | | | | | Day | | | | | | |
| | | [symbol] | [symbol] | 6 | [symbol] | 10 | 12 | 14 | 2 | 3 | 6 | 9 | 16 | 1 [symbol] | 1 [symbol] |
| Cohort | Subject | Concentration (ng/mL) | | | | | | | | | | | | | |
| | N | [symbol] | [symbol] | [symbol] | [symbol] | [symbol] | [symbol] | 9 | 12 | [symbol] | [symbol] | [symbol] | [symbol] | [symbol] | [symbol] |
| | Mean | 0 | 0.0573 | 0.0936 | 0.0855 | 0.0582 | 0.0764 | 0 | 0.592 | 0.805 | 0.883 | 0.999 | 0.816 | 0.651 | 0.529 |
| | SD | 0 | 0. [symbol] | 0.209 | [symbol] .3 [symbol] | 0.2 [symbol] | 0.37 | 0 | 0.493 | 0.681 | 0.6 [symbol] | 0.657 | 0.508 | 0.418 | 0.362 |
| | Min | 0 | [symbol] | [symbol] | [symbol] | [symbol] | 0 | 0 | 0 | 0 | [symbol] | [symbol] .46 | [symbol] | [symbol] | [symbol] |
| | Median | 0 | [symbol] | [symbol] | [symbol] | [symbol] | [symbol] | [symbol] | 0. [symbol] | [symbol] .85 | [symbol] 7 | 0.8 | 8. [symbol] 4 | 0.78 | 0.48 |
| | Max | 0 | [symbol] | [symbol] .56 | [symbol] .4 [symbol] | 8.5 | [symbol] .42 | [symbol] | 3.64 | 3.39 | 3.83 | 3.46 | 1.48 | [symbol] .29 | 0. [symbol] 8 |
| | CV % | | 331.6 [symbol] | 233.52 | 223. [symbol] | 223.62 | 223.5 [symbol] | | 83. [symbol] | 84.51 | 7 [symbol] .7 | 65. [symbol] 9 | 63.24 | 64.17 | 68.4 |
| | Geo- | 2 | | | | | | | | | | 0. [symbol] 64 | | | |
| | metrix | | | | | | | | | | | | | | |
| | Mean | | | | | | | | | | | | | | |

[symbol]

indicates data missing or illegible when filed

Mean concentrations at 8 hours suggest steady state is achieved by Day 3 after multiple dosing of zavegepant in the fasted state
Samples collected during 50 mg QD Fasting Days 1-10, and with Moderate Fat Meal Days 11-14
Pre-dose (CO) concentrations were largely below the LLOQ

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A pharmaceutical formulation in the form of a soft gel dosage form, comprising:
a small molecule calcitonin gene-related peptide (CGRP) receptor antagonist or salt or solvate thereof in an amount 0.01-20 weight % of the total weight of the formulation;
a lipophilic phase comprising triglycerides of fatty acids in an amount of 50-80 weight % of the total weight of the formulation; and
at least one lipophilic surfactant comprising partial esters of polyol and fatty acids in an amount of 10-50 weight % of the total weight of the formulation,
wherein the small molecule CGRP receptor antagonist is zavegepant, ubrogepant, atogepant, telcagepant, or olcegepant, a solvate thereof, or a pharmaceutically acceptable salt thereof.

2. The pharmaceutical formulation according to claim 1, wherein the small molecule CGRP receptor antagonist is zavegepant, a solvate thereof, or a pharmaceutically acceptable salt thereof.

3. The pharmaceutical formulation according to claim 1, wherein the small molecule CGRP receptor antagonist is ubrogepant, a solvate thereof, or a pharmaceutically acceptable salt thereof.

4. The pharmaceutical formulation according to claim 1, wherein the small molecule CGRP receptor antagonist is atogepant, a solvate thereof, or a pharmaceutically acceptable salt thereof.

5. The pharmaceutical formulation according to claim 1, further comprising at least one hydrophilic surfactant with a hydrophilic lipophilic balance (“HLB”) above 10 in an amount of 1-30 wt. % of the total weight of the formulation; and/or wherein the at least one hydrphilic surfactant is present and is selected from the group consisting of polyoxyethylene (20) monooleate, PEG 8 caprylic/capric glycerides, PEG 6 caprylic/capric glycerides, poly(oxyethylene)(4)Lauryl ether and mixtures thereof.

6. (canceled)

7. The pharmaceutical formulation according to claim 1, wherein the triglycerides of fatty acids are medium chain fatty acids.

8. The pharmaceutical formulation according to claim 1, wherein the lipophilic surfactant comprises a mixture of mono and diglyceride of medium chain fatty acids.

9. The pharmaceutical formulation according to claim 1, wherein the formulation does not include water.

10. A method for treatment or prevention of a condition associated with aberrant levels of CGRP in a subject in need thereof, wherein the method comprises administering to the subject a pharmaceutical formulation in a form of a soft gel dosage form, comprising:
a small molecule CGRP receptor antagonist or salt or solvate thereof in an amount 0.01-20 weight % of the total weight of the formulation;
a lipophilic phase comprising triglycerides of fatty acids in an amount of 50-80 weight % of the total weight of the formulation; and
at least one lipophilic surfactant comprising partial esters of polyol and fatty acids in an amount of 10-50 weight % of the total weight of the formulation, wherein the delayed release dosage form is a coated dosage form whose release is pH dependent,
wherein the small molecule CGRP receptor antagonist is zavegepant, ubrogepant, atogepant, telcagepant, or olcegepant, a solvate thereof, or a pharmaceutically acceptable salt thereof.

11. The method according to claim 10, wherein the small molecule CGRP receptor antagonist is zavegepant, a solvate thereof, or a pharmaceutically acceptable salt thereof.

12. The method according to claim 10, wherein the small molecule CGRP receptor antagonist is ubrogepant, a solvate thereof, or a pharmaceutically acceptable salt thereof.

13. The method according to claim 10, wherein the small molecule CGRP receptor antagonist is atogepant, a solvate thereof, or a pharmaceutically acceptable salt thereof.

14. The method according to claim 10, further comprising at least one hydrophilic surfactant with a hydrophilic lipophilic balance (“HLB”) above 10 in an amount of 1-30 wt. % of the total weight of the formulation: and/or wherein the at least one hydrphilic surfactant is present and is selected from the group consisting of polyoxyethylene (20) monooleate, PEG 8 caprylic/capric glycerides, PEG 6 caprylic/capric glycerides, poly(oxyethylene)(4)Lauryl ether and mixtures thereof.

15. (canceled)

16. The method according to claim 10, wherein the triglycerides of fatty acids are medium chain fatty acids.

17. The method according to claim 10, wherein the lipophilic surfactant comprises a mixture of mono and diglyceride of medium chain fatty acids.

18. The method according to claim 10, wherein the formulation does not include water.

19. The method according to claim 10, wherein the condition is a disorder selected from acute migraine, chronic migraine, cluster headache, chronic tension type headache, medication overuse headache, post-traumatic headache, post-concussion syndrome, brain trauma, and vertigo.

20. The method according to claim 10, wherein the condition is a disorder selected from chronic pain, neurogenic vasodilation, neurogenic inflammation, inflammatory pain, neuropathic pain, diabetic peripheral neuropathic pain, small fiber neuropathic pain, Morton's neuroma, chronic knee pain, chronic back pain, chronic hip pain, chronic finger pain, exercise-induced muscle pain, cancer pain, chronic inflammatory skin pain, pain from burns, pain from scars, complex regional pain syndrome, burning mouth syndrome, alcoholic polyneuropathy, chronic inflammatory demyelinating polyradiculoneuropathy, human immunodeficiency virus (HIV) or acquired immunodeficiency syndrome (AIDS)-associated neuropathy, drug-induced neuropathy, industrial neuropathy, lymphomatous neuropathy, myelomatous neuropathy, multi-focal motor neuropathy, chronic idiopathic sensory neuropathy, carcinomatous, neuropathy, acute pain autonomic neuropathy, compressive neuropathy, vasculitic/ischaemic neuropathy, tempero-mandibular joint pain, post-herpetic neuralgia, trigeminal neuralgia, chronic regional pain syndrome, eye pain, and tooth pain.

21. The method according to claim 10, wherein the condition is a disorder selected from non-insulin dependent diabetes mellitus, vascular disorders, inflammation, arthritis, thermal injury, circulatory shock, sepsis, alcohol withdrawal syndrome, opiate withdrawal syndrome, morphine tolerance, hot flashes in men and women, flushing associated with menopause, allergic dermatitis, psoriasis, encephalitis, ischaemia, stroke, epilepsy, neuroinflammatory disorders, neurodegenerative diseases, skin diseases, neurogenic cutaneous redness, skin rosaceousness, erythema, tinnitus, obesity, inflammatory bowel disease, irritable bowel syndrome, vulvodynia, polycystic ovarian syndrome, uterine fibroids, neurofibromatosis, hepatic fibrosis, renal fibrosis, focal segmental glomerulosclerosis, glomerulonephritis, IgA nephropathy, multiple myeloma, myasthenia gravis, Sjogren's syndrome, osteoarthritis, osteoarthritic degenerative disc disease, temporomandibular joint disorder, whiplash injury, rheumatoid arthritis, and interstitial cystitis.

22. (canceled)

23. The method according to claim 10, wherein the condition is a disorder selected from chronic obstructive pulmonary disease, pulmonary fibrosis, bronchial hyperreactivity, asthma, cystic fibrosis, chronic idiopathic cough, and a toxic injury.

24. (canceled)
